(19) United States
(12) Patent Application Publication
(10) Pub. No.: US 2020/0243187 A1
(43) Pub. Date: Jul. 30, 2020

(54) User Interface for Randomized Testing Notification, and Related Methods, Systems, and Software

(71) Applicant: BLA Partners LLC, Burlington, VT (US)

(72) Inventor: Keith Lavoie, South Burlington, VT (US)

(21) Appl. No.: 16/774,953

(22) Filed: Jan. 28, 2020

Related U.S. Application Data
(60) Provisional application No. 62/798,077, filed on Jan. 29, 2019.

(51) Int. Cl. G16H 40/20; H04L 12/58

(52) U.S. Cl. CPC G16H 40/20; G06F 3/0482; H04L 51/24; H04L 51/04

(57) ABSTRACT

Methods, software systems, software, and user interfaces for implementing a mode for randomizing testing of monitored individuals (e.g., substance abusers, mental health patients, etc.), notifying the monitored individuals, and/or tracking the adherence of the monitored individuals to the randomized testing. In some embodiments, time-for-testing (TFT) flags are assigned to the monitored individuals according to one or more assigned testing frequencies. A TFT flag schedule randomization algorithm generates a randomized TFT flag schedule as a function of the testing frequencies and one or more sets of testing-available dates of one or more testing locations. In some embodiments, monitored individuals are required to continually check at least one anonymized random-testing notification user interfaces to determine whether or not their assigned TFT has been called such that they are due for random testing.

### RELATED APPLICATION DATA

This application claims the benefit of priority of U.S. Provisional Patent Application Ser. No. 62/798,077, filed on Jan. 29, 2019, and titled “USER INTERFACE FOR RANDOMIZED TESTING NOTIFICATION, AND RELATED METHODS, SYSTEMS, AND SOFTWARE”, which is incorporated by reference herein in its entirety.

### FIELD OF THE INVENTION

The present invention generally relates to the field of testing notification systems. In particular, the present invention is directed to a user interface for randomized testing notification, and related methods, systems, and software.

### BACKGROUND

Drug testing is becoming more prevalent. For some individuals, testing is ongoing and is best effected if they are monitored on a random schedule so that they do not know when their next sample collection will occur. Currently, randomized-test scheduling is a painstaking manual process.

### SUMMARY

In one implementation, the present disclosure is directed to a method of implementing a randomized testing scheme for notifying a monitored individual of whether or not the monitored individual is due for a monitoring test, wherein the monitored individual has an assigned time-for-testing (TFT) flag selected from a plurality of differing TFT flags and is associated with a testing frequency of a number N times per a time period, the method being executed by a computing system and including determining a set of testing-available dates corresponding to the time period; executing a TFT flag schedule randomization algorithm that uses the testing frequency and the set of the testing-available dates to create a randomized TFT flag schedule having one or more instances of a TFT flag indicator corresponding to the assigned TFT flag, wherein the one or more instances are associated with one or more corresponding randomly selected ones of the testing-available dates; and notifying the monitored individual that the assigned TFT flag has been called.

Additional embodiments of the disclosure include the following.

(1) A randomized scheduling and tracking (RS&T) software system, comprising: an anonymized random-testing (ART) notification user interface (UI) designed and configured to display one or more scheduled time-for-test (TFT) flags and to be available to a plurality of monitored individuals at a general-access location, wherein the one or more scheduled TFT flags are selected from a plurality of TFT flags; a scheduling engine comprising a TFT flag schedule randomization algorithm that randomly schedules the plurality of TFT flags for display by the ART notification UI, wherein each of the plurality of TFT flags is associated with a corresponding testing schedule having a corresponding testing frequency per a corresponding time period, wherein the TFT flag schedule randomizing algorithm randomly schedules each of the plurality of TFT flags as a function of the corresponding testing frequency and the corresponding time period using a pseudorandomization algorithm; and a data-handling engine designed and configured to display a graphical UI (GUI) that allows a monitoring user to assign ones of the plurality of TFT flags to plurality of monitored individuals.

(2) The RS&T software system of (1), wherein the ART notification UI comprises a TFT-display webpage accessible via a uniform resource locator and the TFT-display webpage graphically displays each of the one or more scheduled TFT flags.

(3) The RS&T software system of (2), wherein one or more of the plurality of monitored individuals are assigned to a monitored group having a unique group identifier, the ART notification UI comprising an identification UI configured to receive the unique group identifier and being designed and configured to display the TFT-webpage in response to the identification UI receiving the unique group identifier.

(4) The RS&T software system of (1), wherein the ART notification UI comprises a telephony UI designed and configured to aurally display each of the one or more scheduled TFT flags.

(5) The RS&T software system of (4), wherein one or more of the plurality of monitored individuals are assigned to a monitored group having a unique group identifier, the telephony UI configured to receive the unique group identifier and being designed prior to aurally displaying the one or more scheduled TFT flags.

(6) The RS&T software system of (1), further comprising a system-management engine designed and configured to allow a monitoring user to set up a plurality of monitored groups each having one or more of the plurality of monitored individuals.

(7) The RS&T software system of (6), wherein the system-management engine assigns a unique group identifier to each of the plurality of monitored groups, wherein the unique group identifier allows each of the monitored individuals to access a group-specific TFT flag display via the ART notification UI.

(8) The RS&T software system of (1), further comprising a system-management engine providing a UI for configuring the RS&T software system for use by a plurality of monitoring agencies.

(9) The RS&T software system of (1), further comprising a testing-location UI for tracking ones of the plurality of monitored individuals.

(10) The RS&T software system of (9), further comprising a randomized testing-calendar datastore containing a randomized TFT flag schedule, wherein the testing location UI is designed and configured to display a expected-patient roster based on data in the randomized TFT flag schedule.

(11) A method of enabling substance testing and associated data collection for a plurality of monitored individuals, wherein each monitored individual is assigned a corresponding time-for-testing (TFT) flag from a plurality of predetermined differing TFT flags, the method being performed by a computing system and comprising: assigning the plurality of TFT flags to various ones of a plurality of testing frequencies; executing a TFT flag schedule randomization algorithm to populate a randomized testing-calendar datastore with ones of the plurality of differing TFT flags in accordance with the testing frequencies; for each day populated with at least one of the plurality of differing TFT flags, executing a display algorithm that: determines from the randomized testing-calendar datastore which of the at least one TFT flag to display on an anonymized random-testing (ART) notification user interface (UI); and causes the ART notification UI to display each of the at least one TFT flag.

(12) The method of (11), wherein each of the plurality of testing frequencies has a corresponding time period, and the TFT flag schedule randomization algorithm comprises a pseudorandomization algorithm designed and configured populate the randomized testing-calendar datastore with corresponding respective ones of the plurality of TFT flags for each time period.

(13) The method of (11), further comprising generating and sending an electronic notification message to at least one monitored individuals providing a UI locator that allows the at least one monitored individual to access the ART notification UI.

(14) The method of (13), wherein the ART notification UI comprises a webpage having a uniform resource locator (URL) and the UI locator comprise the URL.

(15) The method of (14), wherein ones of the plurality of monitored individuals are assigned to differing groups, each having a corresponding unique group identifier, the art notification UI requiring each of the plurality of monitored individuals to input the corresponding unique group identifier prior to displaying each of the at least one TFT flag.

(16) The method of (13), wherein the ART notification UI comprises a telephony voice messaging system having a telephone number and the UI locator comprises the telephone number.

(17) The method of (16), wherein ones of the plurality of monitored individuals are assigned to differing groups, each having a corresponding unique group identifier, the art notification UI requiring each of the plurality of monitored individuals to input the corresponding unique group identifier prior to displaying each of the at least one TFT flag.

(18) The method of (11), further comprising presenting to a monitoring user a TFT-flag-assignment UI that permits the monitoring user to assign ones of the plurality of differing TFT flags to ones of the plurality of monitored individuals.

(19) The method of (18), wherein the TFT-flag-assignment UI includes a selector that allows the monitoring user to select from among a plurality of predetermined differing TFT flags.

(20) The method of (18), wherein at least one of the plurality of testing frequencies has at least two of the plurality of differing TFT flags.

A machine-readable storage medium containing machine-executable instructions for performing any one or more of the methods above and/or disclosed elsewhere herein.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1A is a high-level block diagram of an example software as a service (SaaS) deployment of a randomized scheduling and tracking (RS&T) software system made in accordance with aspects of the present disclosure;

FIG. 1B is a high-level block diagram of the RS&T software system of FIG. 1A showing example features and functionalities of the RS&T software system;

FIG. 1C is a flow diagram illustrating an example method of implementing a randomized testing scheme;

FIG. 2 is a diagram illustrating example scheduling periods, along with corresponding assigned time-for-testing (TFT) flags, here, colors, for three groups of monitored individuals;

FIG. 3A is an example of color assignments for three differing testing frequencies;

FIG. 3B is an example week-long randomized TFT flag schedule as determined by a TFT flag schedule randomization algorithm of the present disclosure;

FIG. 3C is an example month-long randomized TFT flag schedule as determined by a TFT flag schedule randomization algorithm of the present disclosure;

FIGS. 4A to 4G illustrate random scheduling of three TFT-flag colors for each of two monitored groups and three testing frequencies;

FIG. 5 is a screenshot of an example monitoring-organization profile page of an example enterprise-based RS&T system;

FIG. 6 is a screenshot of an example window of the example enterprise-based RS&T system that allows a user to set notification preferences and an attendance rule;

FIG. 7 is a screenshot of an example testing-location page 700 of the example enterprise-based RS&T system;

FIG. 8 is a screenshot of an example popup window of the example enterprise-based RS&T system that allows a user to select one or more patient service centers (PSCs) to attach to the enterprise providing the RS&T system;

FIG. 9 is a screenshot of an example testing-center schedule page of the example enterprise-based RS&T system;

FIG. 10 is a screenshot of an example patient-group page of the example enterprise-based RS&T system;

FIG. 11 is a screenshot of an example add-patient-group page of the example enterprise-based RS&T system;

FIG. 12 is a screenshot of another example patient-group page of the example enterprise-based RS&T system;

FIG. 13 is a screenshot of an example patient-group schedule of the example enterprise-based RS&T system;

FIG. 14 is a screenshot of an example upcoming-schedule page of the example enterprise-based RS&T system;

FIG. 15 is a screenshot of an example patients page of the example enterprise-based RS&T system;

FIG. 16 is a screenshot of an example patient-profile page of the example enterprise-based RS&T system;

FIG. 17 is a screenshot of an example attendance history of the example enterprise-based RS&T system;

FIG. 18 is a screenshot of an example patient-assignment-card of the example enterprise-based RS&T system;

FIG. 19 is a screenshot of an example of an expected-patient-roster page of the example enterprise-based RS&T system;

FIG. 20 is a screenshot of an example patient-attendance card of the example enterprise-based RS&T system;

FIG. 21 is a screenshot of an example attendance page of the example enterprise-based RS&T system;

FIG. 22 is a screenshot of another example of a patient-attendance card of the example enterprise-based RS&T system;

FIG. 23 is a screenshot of an example patient-information card of the example enterprise-based RS&T system;

FIG. 24A is a screenshot of an example Colors page of the example enterprise-based RS&T system;

FIG. 24B is a screenshot of an example PIN-entry window of the example enterprise-based RS&T system; and

FIG. 25 is a high-level schematic diagram of an example computing system that can be used to implement any one or more of the aspects of an RS&T system of the present disclosure.

It is noted that any trademarks and copyrighted material appearing in the accompanying drawings are used solely for the sakes of illustration and convenience and not any commercial purpose. Nor should their appearance be considered any sort of endorsement of the trademarks, copyrighted material, underlying products and/or services, or the respective owners. Any trademarks and copyrighted material appearing in the drawings are intellectual property of their respective owners.

### DETAILED DESCRIPTION

I. General Introduction

For the sake of the present disclosure and the appended claims, the following terms shall have the following meanings, as well as any meaning consistent therewith that would be understood by someone skilled in the art.

“Monitored individual”: Person subjected to random testing, such as a medical patient, employee, athlete, prisoner, parolee, etc., among others.

“Monitoring test”: Screening test and/or specimen collection for one or more chemical substances in a monitored individual. Chemical substances include, but are not limited to, prescribed medicaments, illegal substances, or any substance(s) that a monitored individual should or should not have in their body.

“Monitoring user”: Any person authorized to access data on any one or more monitored individuals and/or any one or more monitoring tests via a randomized scheduling and tracking (RS&T) software system (or simply “RS&T system”) of the present disclosure. Examples of monitoring users include, but are not limited to, medical professionals (e.g., physicians, nurses, physicians' assistants, etc.) ordering monitoring tests (and/or their staff), staff members of an agency providing RS&T services using an RS&T system of the present disclosure, and employees of a provider of an RS&T system of the present disclosure, among others.

“Time-for-testing (TFT) flag”: A flag assigned to a monitored individual that indicates when the monitored individual is due for a monitoring test. In some embodiments, a TFT flag denotes that the monitored individual is due for testing the day their TFT flag(s) appear or within another set period of time, such as within a day or two of the TFT being posted. In one example, the TFT flags are colors that may be displayed within a region (e.g., pane, window, page, etc.) on a graphical user interface (GUI) and/or presented in word form (color name) either visually or aurally. Other TFT flags can be used, such as differing animals, differing random images, differing flowers, and differing buildings, among many others.

In many cases, a monitored individual may be assigned one TFT flag at any given time. However, in some cases a monitored individual may be assigned two or more differing TFT flags. For example, a particular monitored individual may have one TFT flag for illicit substance use testing and a different TFT flag for prescribed medicament testing. The testing involved may be at different frequencies and/or may require collection and/or testing by different entities. Monitored individuals may be reassigned a different TFT flag one or more times, such as when they are switched to a different monitored group and/or switched to a different monitoring frequency.

“TFT flag indicator”: A database entry corresponding uniquely to each of the TFT flags. The term “indicator” is used to differentiate, in some embodiments, the database entry from the actual flag. For example, the “indicator” may be a pointer to a flag (e.g., an image) itself In some embodiments, a TFT flag indicator may be the TFT flag itself.

“Anonymized random-testing (ART) notification user interface (UI)” and “notification UI”: Examples of an ART UI include a network-based GUI, such as a web-based GUI and an audio UI, such as a telephone-based dial-in UI, that a monitored individual may access to check for the presence of one or more TFT flags assigned to them to denote that they are due for testing.

“General-access location”: A place where monitored individuals can access the anonymized random-testing notification UI, such as a webpage on a web-server accessed via a uniform resource locator (URL) or on an audio-based system accessed via telephone number, among other things.

“UI locator”: A unique locator that allows a user to access a resource, such as an ART notification UI. A UI locator can be a web URL or phone number, for example.

“Identical access information”: A URL, a telephone number, a URL plus a personal identification number (PIN) or other code, a telephone number plus a PIN or other code, etc., that allows more than one monitored individual to access a general-access location to learn whether or not their TFT flag(s) is/are active.

“Testing-available dates”: Calendar dates that a testing facility is available to receive monitored individuals and conduct monitoring tests. The testing-available dates can be determined in any of a variety of manners, such as by using a testing-location calendar, a testing-location calendar fused with a group calendar, and a testing-location calendar fused with a monitored-user calendar, among others.

“Assigned TFT flag indicators”: TFT flag indicators assigned to various testing-available dates according to a TFT flag schedule randomization algorithm.

“Randomized testing-calendar datastore”: A datastore of any suitable type that stores the assigned TFT flag indicators.

“TFT flag schedule randomization algorithm”: Any algorithm or collection of algorithms that, when executed by a computing system, automatically populate a randomized testing-calendar datastore with TFT flag indicators in a randomized manner. By “randomized” it is meant that the schedule for each TFT flag is effectively unpredictable by a monitored individual assigned to that TFT flag. As those in the fields of certain types of testing know, unpredictability can be the cornerstone to a monitored individual achieving success. For example, in the context of substance-use disorders where a monitored individual is being monitored for use of an illicit substance, when the monitored individual does not know, or cannot predict, when they will be tested, they will be more likely to avoid using the illicit substance. Randomization (unpredictability) can be achieved through any one or more of a variety of techniques, including using a pseudo-randomization algorithm, among others.

“Randomized TFT flag schedule”: The schedule of one or more TFT flags that results from the execution of the TFT flag schedule randomization algorithm one or more times. The randomized TFT flag schedule can be stored in one or more randomized testing-calendar datastores, for example, using TFT flag indicators.

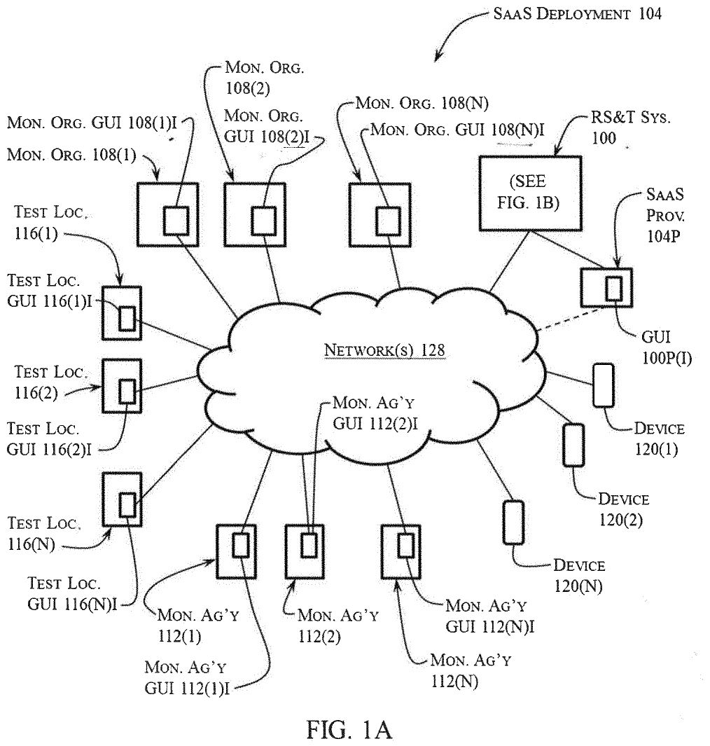

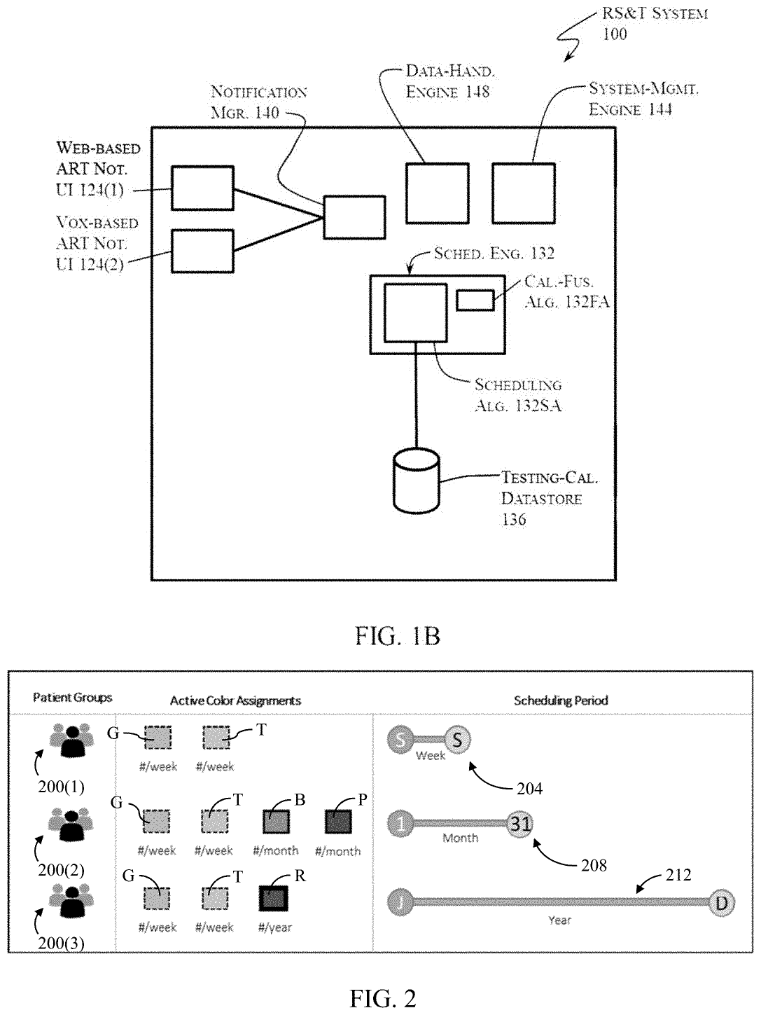

With the foregoing in mind, in some aspects the present disclosure is directed to an RS&T system that automatically randomizes testing schedules for one or more monitored individuals subject to recurrent mandatory testing and that provides features for tracking each individual's adherence to the mandatory testing and for presenting tracked data to one or more monitoring users. Referring now to FIGS. 1A and 1B for illustrative purposes, FIG. 1A shows an example RS&T system 100 in an example deployment, here a software-as-a-system (SaaS) deployment 104, and FIG. 1B shows details of the example RS&T system. In this example, RS&T system 100 is deployed on a suitable computing system, such as one or more web-servers and/or on a distributed computing system (not shown, and is provided and maintained by an RS&T system provider 100P).

It is noted that FIGS. 1A and 1B are provided for the sake of visual illustration of various features and functionalities of an RS&T system of the present disclosure. Those skilled in the art will readily understand that the illustrations of FIGS. 1A and 1B are based on functionalities of various components of the example RS&T system 100 and a useful, but not sole, deployment and are not intended to indicate any particular manner of implementing the RS&T system. Nor are FIGS. 1A and 1B intended to indicate any particular arrangement of software and hardware components. Indeed, those skilled in the art will readily understand that the general features and functionalities depicted in FIGS. 1A and 1B can be implemented in any of a wide variety of computing architectures, such as a SaaS platform architecture (e.g., single-instance, multi-tenant) (as generally depicted by SaaS deployment 104 (FIG. 1B) or a single-instance client-server (e.g., enterprise) architecture (not shown), among others. Fundamentally, the manner in which an RS&T system of the present disclosure, such as RS&T system of FIGS. 1A and 1B, is instantiated is not limited to any particular system architecture. Rather, it is the functionality—not form—that is germane.

For the sake of illustration and not limitation, SaaS deployment 104 (FIG. 1A) is a multitenant deployment in which one or more monitoring organizations 108(1) to 108(N) and zero or more monitoring agencies 112(1) to 112(N) are direct users of the RS&T system 100. (It is noted that the integer variable “N” is used to denote that any number of the corresponding elements can be present. For convenience, the same variable “N” is used for different elements. However, for each element where “N” is used, the value of N need not necessarily be the same as for any other element where “N” is used. For example, when a particular instantiation has a total of 12 monitoring organizations 108(1) to 108(12) (i.e., N=12), this does not mean that there must be 12 monitoring agencies 112(1) to 112(12) (i.e., N=12). Rather, there may any other number of monitoring agencies, such as 2, namely 112(1) and 112(2) (N=2). This holds true for any other elements using the integer variable “N”.)

In this example, each of the monitoring organizations 108(1) to 108(N) is any organization that wants to utilize the randomized scheduling and tracking functionalities of the RS&T system 100, either directly by subscribing to the RS&T system or indirectly by subscribing to one of the monitoring agencies 112(1) to 112(N), if available. Examples of organizations any of the monitoring organizations 108(1) to 108(N) can include, but are not limited to, public and private drug-use intervention organizations, healthcare organizations (e.g., hospitals, hospital networks, outpatient providers, clinics, etc.), companies (e.g., airlines, manufacturers, professional, trucking companies, bus driver providers, etc.), educational organizations (e.g., high schools, colleges, universities, vocational schools, etc.), and sports organizations (amateur teams, profession teams, clubs, etc.), among others, that require at least some of their patients, employees, students, athletes, drivers, etc., to undergo periodic testing.

As alluded to above, each monitoring agency 112(1) to 112(N) is an organization that provides scheduling and tracking services for one or more monitoring organizations, such as any one or more of the monitoring organizations 108(1) to 108(N) mentioned above. In this example SaaS deployment 104, when at least one monitoring agency 112(1) to 112(N) is present, any particular monitoring organization 108(1) to 108(N) may have the option of subscribing directly to RS&T system 100 or indirectly through one of the at least one monitoring agency.

In the example of FIG. 1A, the SaaS deployment 104 involves one or more testing locations 116(1) to 116(N) where the monitored individuals (not shown) go to get tested, e.g., have one or more bodily fluids (e.g., blood, urine, saliva, etc.) and/or other bodily matter (e.g., hair) collected and/or tested. Each of the testing locations 116(1) to 116(N) can be of any one or more types, depending on the entity that owns or manages that testing location. For example, any particular testing location 116(1) to 116(N) may be owned and/or run by an individual monitoring organization 108(1) to 108(N) and may be a test location for only monitored individuals associated with that monitoring organization. As another example, any particular testing location 116(1) to 116(N) may be owned by a particular one of the monitoring agencies 112(1) to 112(N) and may provide testing services for some or all of the ones of the monitoring organizations 108(1) to 108(N) to which that monitoring agency provides RS&T services. As a further example, the provider 100P of the RS&T system 100 may own one or more of the testing locations 116(1) to 116(N) and may allow one or more of the monitoring organizations 108(1) to 108(N) and/or one or more of the monitoring agencies 112(1) to 112(N) to have their monitored individuals use one, some, or all of those testing locations. As still another example, one or more of the testing locations 116(1) to 116(N) may be owned and/or run by one or more other entities, with such testing location(s) providing testing/collections services on a contract basis to any one or more of the monitoring organizations 108(1) to 108(N), monitoring agencies 112(1) to 112(N), and/or provider 100P. Each testing location 116(1) to 116(N) may or may not have the ability to analyze some or all of the samples it collects. Not shown are one or more analysis labs that may analyze the samples collected that are not analyzed at the corresponding testing location 116(1) to 116(N).

In this example, each monitoring organization 108(1) to 108(N) has at least one RS&T organization interface 108(1)I to 108(N)I that allows monitoring users (not shown) within that monitoring organization to access various features and functionalities of the RS&T system 100 necessary/desirable to be accessible to the monitoring organization. In this example, each RS&T organization interface 108(1)I to 108(N)I may be a network browser, such as a web browser, that runs on a suitable computing device (not shown) or computing system (not shown) and that allows a monitored user to connect to various GUIs (e.g., web pages) served by the RS&T system 100, either directly or through one of the monitoring agencies 112(1) to 112(N). In another example, some or all of the RS&T organization interfaces 108(1)I to 108(N)I may be another type of interface, such as an interface provided by a custom software application running on a suitable computing system (not shown) accessible to the monitoring user associated with a corresponding monitoring organization 108(1) to 108(N). Aspects and functionalities that a monitoring user can access via any one or more of RS&T organization interfaces 108(1)I to 108(N)I may include, but not be limited to, aspects and functionalities that allow a monitoring user to: open and/or maintain an account/subscription to the RS&T system 100; add and remove monitored individuals; set up monitored individual parameters; view and edit monitored individual data; add, remove, and modify testing location data; add, remove, and modify test requestor data and/or credentials; add, remove, and modify authorized monitoring user accounts/account data; run, save, and send reports; add, delete, modify scheduling parameters (e.g., frequencies, TFT flags, TFT flag assignments, testing-availability schedules, etc.); and/or perform organizational administrating tasks, among other things, or any subset thereof.

In this example, each monitoring agency 112(1) to 112(N) has at least one RS&T agency interface 112(1)I to 112(N)I that allows monitoring users (not shown) within that monitoring agency to access various features and functionalities of the RS&T system 100 necessary/desirable to be accessible to the monitoring agency. In this example, each RS&T agency interface 112(1)I to 112(N)I may be a network browser, such as a web browser, that runs on a suitable computing device (not shown) or computing system (not shown) and that allows a monitored user to connect to various GUIs (e.g., web pages) served by the RS&T system 100. In another example, some or all of the RS&T agency interfaces 112(1)I to 112(N)I may be another type of interface, such as an interface provided by a custom software application running on a suitable computing system (not shown) accessible to the monitoring user associated with a corresponding monitoring agency 112(1) to 112(N). Aspects and functionalities that a monitoring user can access via any one or more of RS&T agency interfaces 112(1)I to 112(N)I may include, but not be limited to, aspects and functionalities that allow a monitoring user to: open and/or maintain an account/subscription to the RS&T system 100; open and/or maintain accounts for subscribing RS&T monitoring organizations 108(1) to 108(N); add and remove monitored organizations; set up monitored organization parameters; view and edit monitored individual data; add, remove, and modify testing location data; add, remove, and modify test requestor data and/or credentials; add, remove, and modify authorized monitoring user accounts/account data; run, save, and send reports; add, delete, modify scheduling parameters (e.g., frequencies, TFT flags, TFT flag assignments, testing-availability schedules, etc.); and/or perform organizational administrative tasks, among other things, or any subset thereof.

In this example, each testing location 116(1) to 116(N) has at least one RS&T testing-location interface 116(1)I to 116(N)I that allows monitoring users (not shown) within that testing location to access various features and functionalities of the RS&T system 100 necessary/desirable to be accessible to the testing location. In this example, each RS&T testing-location interface 116(1)I to 116(N)I may be a network browser, such as a web browser, that runs on a suitable computing device (not shown) or computing system (not shown) and that allows a monitored user to connect to various GUIs (e.g., web pages) served by the RS&T system 100. In another example, some or all of the RS&T testing-location interfaces 116(1)I to 116(N)I may be another type of interface, such as an interface provided by a custom software application running on a suitable computing system (not shown) accessible to the monitoring user associated with a corresponding testing location 116(1) to 116(N). Aspects and functionalities that a monitoring user can access via any one or more of RS&T testing-location interfaces 116(1)I to 116(N)I may include, but not be limited to, aspects and functionalities that allow a monitoring user to: view and edit monitored individual data; add, remove, and modify testing location data; add, remove, and modify test requestor data and/or credentials; add, remove, and modify authorized monitoring user accounts/account data; run, save, and send reports; add, delete, modify scheduling parameters (e.g., frequencies, TFT flags, TFT flag assignments, testing-availability schedules, etc.); and/or enter and modify information regarding testing of monitored individuals (e.g., test-performance flag, test results, tester notes, etc.), among other things, or any subset thereof.

The RS&T system provider 100P has one or more suitable RS&T-provider interfaces 100P(I) that allows monitoring users (not shown) within the RT&T system provider to access various features and functionalities of the RS&T system 100 necessary/desirable to be accessible to the RS&T service provider. In this example, RS&T-provider interface 100P(I) may be a network browser, such as a web browser, that runs on a suitable computing device (not shown) or computing system (not shown) and that allows a monitored user to connect to various GUIs (e.g., web pages) served by the RS&T system 100. In another example, the RS&T-provider interface 100P(I) may be another type of interface, such as an interface provided by a custom software application running on a suitable computing system (not shown) accessible to the monitoring user associated with the RS&T provider 100P. Aspects and functionalities that a monitoring user can access via the RS&T-provider interface 100P(I) may include, but not be limited to, aspects and functionalities that allow a monitoring user to: open and/or maintain an account/subscription to the RS&T system 100; open and/or maintain accounts for subscribing RS&T monitoring organizations 108(1) to 108(N); add and remove monitored organizations; set up monitored organization parameters; open and/or maintain accounts for subscribing RS&T monitoring agencies 112(1) to 112(N); add and remove monitored agencies; set up monitored agency parameters; view and edit monitored individual data; add, remove, and modify testing location data; add, remove, and modify test requestor data and/or credentials; add, remove, and modify authorized monitoring user accounts/account data; run, save, and send reports; add, delete, modify scheduling parameters (e.g., frequencies, TFT flags, TFT flag assignments, testing-availability schedules, etc.); and/or perform administrative tasks, among other things, or any subset thereof.

The SaaS deployment 104 of this example also involves a plurality ART notification UI access devices 120(1) to 120(N), which may correspond, respectively, to a plurality of monitored individuals. Each ART notification UI access device 120(1) to 120(N) allows a monitored individual to access at least one ART notification UI, here, either the web-based ART notification UI 124(1) or the voice-based ART notification UI 124(2), or both. To access the web-based ART notification UI 124(1) in this example, any of the ART notification UI access devices 120(1) to 120(N) will typically require a web browser (not shown) or dedicated software app (not shown). In these cases, such ART notification UI access devices 120(1) to 120(N) will be some sort of computing device, such as a smartphone, tablet computer, laptop computer, or a desktop computer, among others. To access the voice-based ART notification UI 124(2) in this example, which is accessible via voice telephony, any of the ART notification UI access devices 120(1) to 120(N) will require the ability to communicate via voice telephone. In this case, such ART notification UI devices 120(1) to 120(N) will be a telephony device, such as a smartphone, mobile phone, landline phone, or a computing device running software configured to allow a user to connect to the voice-message-base ART notification UI 124(2). Examples of such software include a voice-over-Internet-protocol (VOIP) software app or a web-browser that allows a user to connect to a web-based VOIP service, among others.

As those skilled in the art will readily appreciate, the SaaS deployment 104 may be effected by the various components, for example, the RS&T system 100, the monitoring organizations 108(1) to 108(N), the monitoring agencies 112(1) to 112(N), the testing locations 116(1) to 116(N), and the ART-notification-UI access devices 120(1) to 120(N), being connected to one or more networks (collectively illustrated by network(s) 128 in FIG. 1A) that may include, but not be limited to, the Internet (or other global network), one or more local-area networks (LANs) (e.g., Ethernet LANs and/or wireless LANs), one or more personal-area networks (PANs), and/or one or more cellular networks, or any combination thereof, among others. Fundamentally, there are no limitations on the network(s) 128 other than it/they enable functionality of the RS&T system 100 described herein.

As noted above, the RS&T system 100 includes one or more ART notification UIs (hereinafter, simply “notification UIs”) (here, web-based notification UI 124(1) and voice-based notification UI 124(2) that allow some or all monitored individuals (not shown) to determine whether or not they are due to be tested during any particular testing period (e.g., a day, a week, etc.). As also noted above, randomized testing provides a measure of uncertainty for a monitored individual that can give them incentive to either abstain from one or more illicit substances or maintain a certain regimen of taking a prescribed medication, as the case may be. Details of example embodiments of the web-based and voice-based notification UIs 124(1) and 124(2) are presented below.

In some embodiments, each monitored individual is advised that they are to be randomly tested at a particular frequency, such as 3 times per week, 2 times per week, one time per week, once every two weeks, once a month, N times in M months, once a year, etc., depending on that monitored individual's condition. In these embodiments, the software (i.e., the machine-executable instructions) (not shown) of the RS&T system 100 may be configured with some or all of these testing frequencies, such that when a monitoring user (not shown) inputs a new monitored individual into the RS&T system, the monitoring use can select the assigned frequency either directly or indirectly by assigning a TFT flag (not shown) to that monitored individual. Detailed examples of assigning one or more testing frequencies to each monitored individual are presented below.

In this embodiment, each TFT flag is unique relative to all of the other TFT flags. For example, if the TFT flags are colors, no two colors are the same. Depending, for example, on the number and nature of the monitored individuals and/or the number of testing locations available, among other things, each testing frequency may have more than one assigned TFT flag. For example, and as will become apparent from the description of the example TFT flag schedule randomization algorithm presented below, a testing frequency may have multiple TFT flags, for example, to avoid having too many monitored individuals assigned to a single TFT flag, as this could cause one or more testing locations to become overburdened with monitored individuals on days that such TFT flag is scheduled. As another example, for spouses in a household, or more generally, multiple individuals living together, that are required to be randomly tested at the same frequency, it can be beneficial to assign those monitored individuals to differing TFT flags. As a further example, differing groups of monitored individuals may be assigned differing colors for the same frequency. Those skilled in the art will readily appreciate that any testing frequency may have multiple differing TFT flags for any two or more of the reasons of these examples, among other reasons. A monitored individual can be assigned to a testing frequency indirectly by assigning a specific TFT flag to that individual, the TFT flag having already been assigned within the RS&T system to a particular testing frequency. Examples are described below.

Each notification UI 124(1) and 124(2) may be any suitable notification UI that allows monitored individuals to access it to determine whether or not their assigned TFT flag(s) is/are present. In some embodiments, the RS&T system 100 may be set up so that it updates each notification UI 124(1) and 124(2) overnight or at another predetermined time. In such cases, each monitored individual may be instructed to check one or the other, or either, of the notification UIs 124(1) and 124(2) daily within a particular timeframe that is after the notification UI has been updated and before all testing locations reachable by the monitored individual close. In some embodiments, either or each of the notification UIs 124(1) and 124(2) may reside at a suitable general-access location (not shown), such as on a web server accessible via a URL (e.g., for web-based notification UI 124(1), on a voice messaging system accessible via a telephone number (e.g., for voice-based notification UI 124(2), or on a short messaging service (SMS) system accessible via an SMS identifier, among others. In each of these cases, each monitored individual accesses the notification UI 124(1), 124(2) using identical access information that is the same access information given to all other monitored individuals or all other monitored individuals within a particular monitored group (see below). This identical access information may be a URL, a telephone number, an SMS identifier, a URL plus a PIN or other code, a telephone number plus a PIN or other code, an SMS identifier plus a PIN or other code, etc.

As described below, in some embodiments the RS&T system 100 may be designed and configured to manage multiple groups of monitored individuals (hereinafter “monitored groups”), each of which can have all monitored individuals assigned to more than one frequency or to only a single frequency. Such monitored groups may be defined by any one or more of a number of factors. For example, monitored groups may be set up for an individual company, a government agency, a university, and/or other organization (e.g., sports team, school bus drivers for a specific school district, etc.), that require at least some of their employees, students, athletes, drivers, etc., to undergo periodic testing. In addition or alternatively, monitored groups may be set up for individual healthcare providers, testing agencies, and government entities (e.g., department of corrections, etc.), among others, and any combination thereof.

When the RS&T system 100 of FIGS. 1A and 1B is set up to handle multiple monitored groups, each notification UI 124(1) and 124(2) may be configured to limit access of the TFT flag(s) by monitored groups. For example, in the web-based notification UI 124(1), each monitored group may be given a monitored-group-unique URL, and in the voicemail-message-based notification UI 124(2), each monitored group may be given a unique telephone number or extension. As another example, in the web-based notification UI 124(1), all monitored groups may use the same URL but enter differing PINs or other codes. Similarly, in the voicemail-message-based notification UI 124(2), all monitored groups may use the same general telephone number but enter differing monitored-group-unique PINs or other codes once in the voicemail systems. Many other possibilities exist for permitting access on a group-by-group basis.

As noted above, an important feature of an RS&T system of the present disclosure, such as RS&T system 100 of FIGS. 1A and 1B, is the ability to automatically randomize testing schedules for the monitored individuals active within the RS&T system. Consequently, in the example embodiment shown in FIGS. 1A and 1B the RS&T system 100 includes a scheduling engine 132 (FIG. 1B) that executes a TFT flag schedule randomization algorithm 132SA configured to automatically randomize and schedule testing days for the active monitored individuals using TFT flags that anonymize the scheduling on the monitored-individual side of the RS&T system 100. In some embodiments, the TFT flag schedule randomization algorithm 132SA may perform the randomization using a suitable pseudorandom number generator (PRNG), such as the Mersenne Twister PRNG well known in other computing fields, among many others. The specific implementation of any PRNG used is not necessarily critical to the randomized scheduling; rather, it is the automation of the randomization process that provides value and usefulness to the RS&T system 100.

In some embodiments, the automatic scheduling of test days involves configuring the scheduling engine 132 (FIG. 1B) to execute the TFT flag schedule randomization algorithm 132SA continually at one or more specified times or at certain intervals. For example, the scheduling engine 132 may run the TFT flag schedule randomization algorithm 132SA overnight either every day of the week or only on the nights before each day that has at least one testing location 116(1) to 116(N) (FIG. 1A) open for testing. Other schedules on which the scheduling engine 132 can execute the TFT flag schedule randomization algorithm 132SA can be used if desired. For example, the scheduling engine 132 may be configured to execute the TFT flag schedule randomization algorithm 132SA only once or twice a week. However, with less frequent executions the TFT flag scheduling algorithm would not pick up any changes made between the executions, such as changes of assignments to TFT flags, changes to schedules, and/or additions or removals of monitored individuals to or from active status, among others, on a day-to-day basis. Therefore if the RS&T system 100 posts TFT flag updates daily, such changes would not be reflected, for example, in the daily TFT flags that the notification UIs 124(1) and 124(2) make available to monitored individuals. However, this may be acceptable under specific circumstances.

In some embodiments, the TFT flag schedule randomization algorithm 132SA may be configured to perform scheduling operations one monitored group at a time. In some embodiments, each individual monitored group may have its own monitored-group schedule (not shown) that indicates the day(s) on which its members, i.e., its assigned monitored individuals, are supposed to be available for testing. Differing monitored groups may have differing randomized TFT flag schedules for any of a variety of reasons, such as types of monitored individuals being monitored (e.g., parolees versus athletes), observance of differing sets of holidays, and differing observances of weekend days, among others. Consequently, in this example the TFT flag schedule randomization algorithm 132SA may account for these differing randomized TFT flag schedules when scheduling testing days for the differing monitored groups.

The testing locations 116(1) to 116(N) may similarly have their own availability schedules (not shown) of when they are open to monitored individuals for testing. Consequently, the TFT flag schedule randomization algorithm 132SA may be configured to account for individual testing-location availability schedules. In some embodiments, the TFT flag schedule randomization algorithm 132SA may also be configured to account for availability schedules of individual ones of the monitored individuals. That said, it is noted that the variability in availability schedules of the monitored individuals can be accounted for in other ways, such as by individually excusing monitored individuals if they miss a scheduled testing and can provide adequate proof of their unavailability.

When accounting for two or more availability schedules, the TFT flag schedule randomization algorithm 132SA may include a calendar-fusion algorithm 132FA that generates an effective testing schedule from two or more availability schedules. As a simple example, for a testing location open seven days a week and a monitored group having a Monday-through-Friday testing-availability schedule, the effective testing schedule resulting from the calendar-fusion algorithm 132FA fusing the two availability schedules for that monitored group and that testing location would indicate that testing can be scheduled only on Mondays, Tuesdays, Wednesdays, Thursdays, and Fridays. The dates that are available for testing based on an effective testing schedule are referred to herein and in the accompanying claims as “testing-available dates.”

Below in the next section is an example TFT flag schedule randomization algorithm that generates a testing schedule by serially processing a plurality of monitored groups. This example TFT flag schedule randomization algorithm may be used for the TFT flag schedule randomization algorithm 132SA of FIG. 1B. Those skilled in the art will appreciate that the precise TFT flag schedule randomization algorithm described herein is merely exemplary and that many variations of this TFT flag schedule randomization algorithm and other automatic TFT flag schedule randomization algorithm are possible.

The scheduling engine 132 may store the results of running the TFT flag schedule randomization algorithm 132SA in a randomized testing-calendar datastore 136 (FIG. 1B), which may be any type of datastore suitable for representing the calendared TFT flags. In one embodiment, the randomized testing-calendar datastore 136 may be a table (not illustrated) that includes a date column, indicating a plurality of days in the scheduling periods of the differing frequencies (differing TFT flags), and a plurality of TFT-flag columns, one for each possible TFT flag in the RS&T system 100. For each day in the date column, the assignment of a TFT flag to that day is indicated by the presence of a TFT flag indicator in a flag field of corresponding TFT flag column. In another embodiment, the randomized testing-calendar datastore 136 may include a date column, indicating a plurality of days, and an active-flags column. For each day in the date column, a corresponding active-flag field in the active-flag column may contain a unique TFT flag indicator for each flag assigned to that day. If there are no TFT flags assigned to a particular day, the corresponding active-flag field will be empty. In some embodiments, each TFT flag indicator may be a unique code for the corresponding TFT flag and the RS&T system 100 may include a table that relates the TFT flag indicators to digital files containing the corresponding respective TFT flags (e.g., images, etc.) themselves. In some embodiments, each TFT flag indicator may itself be a pointer to a corresponding digital file containing the corresponding TFT flag. Of course, other types of datastores may be used for the randomized testing-calendar datastore 136 and other ways of arranging and populating the randomized testing-calendar datastore may be used.

In addition to the scheduling engine 132, the RS&T system 100 may also include a notifications manager 140 that manages the one or more notification UIs, here, notification UIs 124(1) and 124(2). For example, immediately after the scheduling engine 132 has run the TFT flag schedule randomization algorithm 132SA (e.g., overnight each day), the notifications manager 140 may update each of the notification UIs 124(1) and 124(2). For example, if the web-based notification UI 124(1) is composed of one or more webpages (not shown), the notifications manager 140 may read the entries of the randomized testing-calendar datastore 136 for the current day and update the TFT flag(s) displayed on the webpage(s). In a simple form, the notifications manager 140 may be built into each webpage such that it continually accesses the randomized testing-calendar datastore 136 and displays whatever TFT flag(s) are contemporaneously in the randomized testing-calendar datastore for the current day. As another example, for the voice-based notification UI 124(2) the notifications manager 140 may update, switch-out, etc., the recording(s) played when a monitored individual calls into the notification UI. The notifications manager 140 may be implemented any useful way that updates each of the notification UIs 124(1) and 124(2) following the TFT flag schedule randomization algorithm 132SA updating the randomized testing-calendar datastore 136.

The RS&T system 100 may also include a system-management engine 144 that allows the RS&T system provider 100P to set up the RS&T system for any number of monitored individuals (not shown), any number of organizations (e.g., monitoring organizations 108(1) to 108(N) and/or monitoring agencies 112(1) to 112(N)), any number of monitored groups (not shown), and/or any number of testing locations (e.g., testing locations 116(1) to 116(N). The RS&T system provider 100P may have access to system-management engine 144 through the one or more RS&T system provider interfaces 100P(I). Examples of features that the system-management engine 144 may have are illustrated below in a subsequent section of this disclosure. As exemplified below, the system-management engine 144 is implemented using web-based system-management GUIs that are particularly suited to the cloud-based implementation of SaaS deployment 104. In non-web-based embodiments, similar system-management GUIs may be provided as non-web-based system-management GUIs. Those skilled in the art will readily understand how to provide system-management UIs suitable for the particular implementation at issue.

The RS&T system 100 may also include a monitored-individual data-handing engine 148 that provides various data-handling GUIs that allow system users, such as monitoring users in the monitoring organizations 108(1) to 108(N), monitoring agencies 112(1) to 112(N), and testing locations 116(1) to 116(N), to view data concerning one or more monitored individuals and/or enter data concerning one or more monitored individuals, among other things. The monitoring organizations 108(1) to 108(N), monitoring agencies 112(1) to 112(N), and testing locations 116(1) to 116(N) may have access to monitored-individual data-handling engine 148 via corresponding respective ones of RS&T organization interface(s) 108(1)I to 108(N)I; RS&T agency interface(s) 112(1)I to 112(N)I, and RS&T testing-location interface(s) 116(1)I to 116(N)I. Examples of data-handing GUIs that the monitored-individual data-handling engine 148 may provide are illustrated below in a subsequent section of this disclosure. Like any system-management GUIs provided, each data-handling GUI may or may not be web-based, though the examples below are web-based. The monitored-individual data-handling engine 148 may provide tracking functionalities of the RS&T system 100, and one or more monitored-individual data-handling GUI may allow the monitoring users to view various statuses of one, some, or all of the monitored individual and/or one or more monitored groups, among other functionalities.

FIG. 1C illustrates an example method 170 of implementing a randomized testing scheme in accordance with the present disclosure. The testing scheme can be used for notifying a monitored individual of whether or not that monitored individual is due for a monitoring test. In this example, the monitored individual has an assigned TFT flag selected from among a plurality of differing TFT flags. The assigned TFT flag is associated with a testing frequency of a number N times per time period. For the sake of convenience, an example of method 170 is described in the general context of RS&T system 100 of FIGS. 1A and 1B. However, those skilled in the art will readily appreciate that the method 170 can be performed by another suitable configured RS&T system made in accordance with aspects of the present disclosure.

Referring to FIG. 1C, and also to FIGS. 1A and 1B and other figures as noted, the method 170 includes, at block 175, a suitable computing system (not shown) determining a set of one or more testing-available dates corresponding to the time period of the testing frequency of the assigned TFT flag. As discussed herein, a set of testing available dates can be determined in any of a variety of ways. For example, an algorithm can be used to use the time period (e.g., week, month, year, etc.) to determine the range of possible dates (e.g., a calendar week, or remaining portion thereof, for a frequency having a time period of a week). Optionally, this range of possible dates can be fused with one or more testing-location availability calendars and/or an availability calendar of the monitored individual, which may be a group-availability calendar if the monitored individual is assigned to a group. Fusing of calendars to determine testing-available dates to which TFT flags can be randomly assigned may be performed by the calendar-fusion algorithm 132FA. Examples of fusing calendars are described below in section IV.D.2.

At block 180, the computing system executes a TFT flag schedule randomization algorithm, such as the TFT flag schedule randomization algorithm 132SA (FIG. 1B) executed by the calendaring engine 132 (FIG. 1B). The TFT flag schedule randomization algorithm may use the testing frequency and the testing-available dates within the timer period to create a randomized TFT flag schedule having one or more instances of a TFT flag indicator corresponding to the assigned TFT flag, with the one or more instances being associated with one or more corresponding randomly selected one of the testing available dates. Randomization may be provided using a suitable pseudorandomization algorithm, nonlimiting examples of which are described herein. The randomized TFT flag schedule may be stored in a suitable testing-calendar datastore, such as testing-calendar datastore 136 of FIG. 1B.

At block 185, the computing system notifies the monitored individual that the assigned TFT flag has been called. The notification that occurs at block 185 can be direct and/or indirect, depending on the desired mode(s) of notification. An example of a direct notification at block 185 is the sending, by the computing system, of an electronic message (e.g., direct message, email message, voicemail message, etc.) to the monitored individual notifying the monitored individual that their assigned TFT has been called and, therefore, that they are due for testing. In an RS&T software system in which testing is based on a daily system (i.e., due TFT flags are updated daily (e.g., work week, full week, etc.), the electronic messaging may be initiated by an algorithm checking (e.g., daily) the randomized TFT flag schedule to determine which, if any, assigned TFT flags have been called (e.g., by determining whether or not and which TFT flag indicator(s) are present in the randomized TFT flag schedule for that day). For each TFT flag indicator present in the randomized TFT flag schedule for the current day, the computing system may access a list that correlates the monitored individuals with their assigned TFT flag to determine which monitored individual(s) need to be notified that the computing system has called their assigned TFT flag and that they are now considered due for testing. Such a direct notification may include any suitable information, such as a verbal message that they are due for testing and/or a display of their assigned TFT flag, among other things.

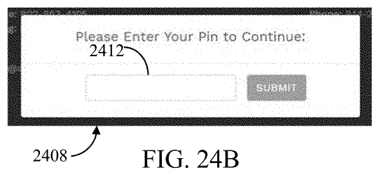

An example of indirect notification is the utilization of one or more ART notification UIs to display the assigned TFT flag to the monitored individual. Examples of ART notification UIs include, but are not limited to, web-based ART notification UI 124(1), voice-based ART notification UI 124(2) (FIG. 1B), and the ART notification UI examples discussed below, for example, in section IV.G. In the case of a web-based ART notification UI, such as web-based ART notification UI 124(1) of FIG. 1B, the computing system may update a webpage (see, e.g., Colors page 2400 of FIG. 24) each day so as to display the TFT flag(s), if any, that the computing system has called for that day (e.g., by using the TFT flag schedule randomization algorithm). As described in more detail elsewhere herein, the monitored individual may access the webpage to see whether or not the computing system has called their assigned TFT flag. A voice-based ART notification UI, such as voice-based ART notification UI 124(2) of FIG. 1B, may work generally similarly to a web-based ART notification UI, except for the means by which a monitored individual accesses it and how the assigned TFT flag is presented. As mentioned above, requiring the monitored individual to check an ART notification UI daily (or on some other schedule) can contribute to success of the monitored individual.

II. Example TFT Flag Schedule Randomization Algorithm

In this example embodiment of a TFT flag schedule randomization algorithm, two scheduling groups are used: Scheduling Group A for monitored groups having existing randomized TFT flag schedules that do not require change, and Scheduling Group B for monitored groups either without existing randomized TFT flag schedules or that need new randomized TFT flag schedules due to changed circumstances, such as, for example, a change in a testing-location availability calendar or a monitored-group availability calendar, or both, removal of a group, removal or addition of a frequency, etc. Monitored groups having existing randomized TFT flag schedules that do not require changes retain their current schedules.

Also in this embodiment, the TFT flag schedule randomization algorithm is configured to handle multiple organizations, multiple groups per organization, multiple frequencies per monitored group, and multiple TFT flags per monitored group. At a high level, this example TFT flag schedule randomization algorithm determines the scheduling needs of an organization (e.g., testing-location and group schedules), then proceeds at the monitored-group level, and then at the TFT-flag level. The TFT flag schedule randomization algorithm handles each organization and monitored group independently of, respectively, the other organizations and monitored groups.

In this embodiment, the scheduling engine (e.g., scheduling engine 132 of FIG. 1B) executes this TFT flag schedule randomization algorithm overnight every day of the week. In this manner, all changes made during the previous day will affect all future scheduling of TFT flags, and any scheduling changes resulting from the changes will be effective beginning the following day. In other instantiations, the scheduling engine may be configured to execute the TFT flag schedule randomization algorithm on another schedule, which may be selected by monitoring user or other user of the RS&T system.

II.1. Categorizing Monitored Groups

In this example embodiment, the TFT flag schedule randomization algorithm determines which of Groups A and B, above, monitored groups belong in as follows:
1) For each of the testing frequencies available, find the start and end dates for the period of that testing frequency, starting with the beginning date of the scheduling process (typically today). In this example, weeks begin on Sundays and end of Saturdays. Months and years are by the calendar.
2) Categorize each of the monitored groups based on whether they need TFT-flag schedules for any of the testing frequencies used in that monitored group. In this example, the TFT flag schedule randomization algorithm uses the following steps to determine the statuses of the TFT-flag schedules:
   a) Find all TFT flags assigned to the current testing frequency that have an existing randomized TFT flag schedule for days within the current testing-frequency period. Place monitored groups not having any such entries into Schedule Group B.
   b) Determine if there is at least one TFT flag that needs an updated schedule. A TFT flag needs an updated schedule if it currently has a schedule but no longer has active monitored individuals associated with it or if it does not currently have a schedule and now has active monitored individuals. Place each monitored group that has at least one TFT flag that needs an updated randomized TFT flag schedule into Schedule Group B.
   c) Find the last time a randomized TFT flag schedule was updated for any of the frequency TFT flags that currently have randomized TFT flag schedules.
   d) If the current monitored group's availability schedule was updated after the time that the frequency TFT flag was last scheduled, then put the current monitored group into Schedule Group B.
   e) If the testing-available schedule for any of the testing locations associated with the current patient group was updated after the time that the frequency TFT flag was last scheduled, then put the current monitored group into Schedule Group B.
   f) Put all other monitored groups into Schedule Group A.

II.2. Scheduling Monitored Groups

Once the TFT flag schedule randomization algorithm has categorized the monitored groups based on whether or not their randomized TFT-flag schedules need updating, it can begin scheduling the monitored groups requiring scheduling. It is noted that in this example, the TFT flag schedule randomization algorithm is configured to schedule TFT flags that need scheduling during each current period, or remainder thereof, for each testing frequency (e.g., current week for a weekly frequency) plus the next period. Thus, for a weekly testing frequency, the TFT flag schedule randomization algorithm considers the current week or remainder thereof if the night the scheduling engine is running the TFT flag schedule randomization algorithm any day of the week other than Sunday (see above for the definition of a week) plus the full week following the current week or its remainder.

For the monitored groups in Schedule Group A, the TFT flag schedule randomization algorithm does not make any changes. For monitored groups in Schedule Group B, the TFT flag schedule randomization algorithm proceeds as follows:
1) Determine the range of dates available to be scheduled for each testing frequency based on the period of the testing frequency:
   a) The date range for a testing frequency of N times per week is the remainder of the current week plus next week.
   b) The date range for a testing frequency of N times per month is the remainder of the current calendar month plus the next calendar month.
   c) The date range for a testing frequency of N times per M months is calculated by dividing the months of the year by M to create sets of months. The date range is the remainder of the current set plus the next set. For example, if the testing frequency is one time every two months, then there are 12/2=6 sets of two months in the year. If the current day that the scheduling engine is running the calendaring algorithm is May 20^{th}, then the date range is May 20^{th} to June 30^{th} (3^{rd} 2-month set of the year) plus July 1^{st} to August 31^{st} (4^{th} 2-month set of the year) for a total of May 20^{th} to August 31^{st}.
   d) The date range for a testing frequency of N times per year is the remainder of the current year plus next year.
2) For the monitored groups in Schedule Group B: for each day in the date ranges for the various testing frequencies, count the number of TFT flags scheduled. These are the base values used when doing load balancing to ensure that testing capabilities are not overtaxed.
3) For each testing frequency, starting with the fastest (here, three times per week) and working down to the slowest (here, once per year):
a) For each monitored group that allows for the current testing frequency (i.e., those that allow all frequencies and those having the current testing frequency):
   i) Get the effective schedule within the current frequency date range for the monitored group by fusing the monitored group's availability schedule with the testing-available schedules for all of the testing locations available to the current monitored group.
   ii) Determine which of the current frequency's TFT flags that the scheduling engine needs to schedule for the monitored group as follows:
      (1) Based on the day the scheduling engine executes the TFT flag schedule randomization algorithm (i.e., the current day) and for a current TFT flag, count the number of days scheduled in the past within the full period of the current frequency (e.g., current week, month, year, etc.) and determine the number of days remaining to be scheduled:
         (a) If there are no remaining days to be scheduled, clean out the further schedule for the TFT flag beyond the date range and mark the TFT flag as done.
         (b) If the current day is in the middle of the period of the current frequency, compute a value equal to the frequency number of testing times, multiplied by the number of days remaining in the full period divided by the total number of days in the full period. Add 0.25 to the result and round down to the nearest whole number, with a minimum of one. Use the smaller of the computed value and the number of days remaining for the number of days to be scheduled.
         (c) If there are remaining days to be scheduled, mark the current TFT flag as needing that number of remaining days scheduled.
      (2) Sort the TFT flags that still have remaining days to be scheduled so that those needing the most days scheduled will be processed first.
         (a) While there are still TFT flags with remaining days to be scheduled, for each of the TFT flags to be scheduled:
            (i) Maintain a subset of the days remaining that could be scheduled that does not include days within a minimum interval of an existing scheduled day. The interval is computed as a percentage of the frequency period (example default: 5%). Use this subset as long as it contains days, and then switch to the regular days remaining that could be scheduled. This results in two groups of available days being maintained by the calendaring algorithm, namely:
            1. all remaining days in the period that meet the minimum days between collections, and
            2. all remaining available days in the period.
            (ii) Choose one of the days in the subset that has the fewest other TFT flags to be scheduled. In one example, the TFT flag schedule randomization algorithm uses a PRNG to make the choices. An example illustrating this step is described below.
            (iii) Remove the chosen day from the list of available days remaining for the current TFT flag.
            (iv) Add the chosen day to the schedule for the current TFT flag.
            (v) For the chosen day, increment by one the count of TFT flags scheduled for that day by one.
            (vi) Decrease by one the count of the number of times that the current TFT flag still needs to be scheduled.
            (vii) If the current TFT flag still has at least one time that needs to be scheduled, add the current TFT flag to a list of TFT flags to be scheduled in the next pass.

As noted above, the choosing that the TFT flag schedule randomization algorithm performs at 3a(2)(ii) can be implemented using a PRNG, such as a Marsenne Twister PRNG, among others. So long as there are days in the first group noted above at 3a(2)(i), the TFT flag schedule randomization algorithm will choose one of those days at random using the PRNG. If there are no days left in the first group, then the TFT flag schedule randomization algorithm chooses one of the days in the second group using the PRNG. In some embodiments, these first and second groups can be zero-based collections of days (i.e., the index of the first entry in each group is 0, the last entry has an index of the number of days in the group minus one). The TFT flag schedule randomization algorithm picks the index of the day to use by getting an integer value between 0 and the number of days in the group at issue minus one using the PRNG. Following is an example illustrating this process.

This example uses a one week period with two TFT flags needing three days each scheduled, with a minimum of two days between tests for each TFT flag. For this example, the days of the week are shown in the lists, with Sunday being day 0 and Saturday day 6. In other embodiments, the values stored may be the dates of the days (so, one might see Jan. 1, 2019, rather than 0). To begin, the TFT flag schedule randomization algorithm creates the following lists:

TFT Flag 1

Group 1 = [0,1,2,3,4,5,6]
Group 2 = [0,1,2,3,4,5,6]
Schedule = [ ]

TFT FLAG 2

Group 1 = [0,1,2,3,4,5,6]
Group 2 = [0,1,2,3,4,5,6]
Schedule = [ ]

Used Days = [ ]

Choose a day for TFT Flag 1. No days have previously been scheduled, so all remaining days can be considered. Since there are seven entries in TFT Flag 1's group 1, the calendaring algorithm uses the PRNG to generate a number between 0 and 6. Say the PRNG chooses 2 (day 2). The calendaring algorithm removes that entry from each of TFT Flag 1's groups. In addition, the TFT flag schedule randomization algorithm removes days within two days of that entry from group 1. Day 2 now has one TFT flag scheduled. The modified collections look like this:

Color 1

Group 1 = [5,6]
Group 2 = [0,1,3,4,5,6]
Schedule = [2]

Used Days = [[2,1]]

Choose a day for TFT Flag 2. Since day 2 has been scheduled and no other days have been scheduled, the calendaring algorithm removes day 2 from consideration by generating a subset of TFT Flag 2, Group 1, that does not contain day 2. That subset looks like [0,1,3,4,5,6]. The TFT flag schedule randomization algorithm uses the PRNG to generate a number between 0 and 5. For the sake of example, say the PRNG chooses 2 (i.e., day 3). After the updates, the changed collections are:

Color 2

Group 1 = [0, 6]
Group 2 = [0,1,2,4,5,6]
Schedule = [3]

Used Days = [[2,1], [3,1]]

Choose a second day for TFT Flag 1. Only two days remain in its Group 1. Neither of them has been scheduled yet, so the TFT flag schedule randomization algorithm can choose either. The TFT flag schedule randomization algorithm uses the PRNG to select a value of either 0 or 1. Say it chooses 1 (day 6). The modified collections are:

Color 1

Group 1 = [ ]
Group 2 = [0,1,3,4,5]
Schedule = [2,6]

Used Days = [[2,1], [3,1], [6,1]]

Choose a second day for TFT Flag 2. Only two days are in its Group 1, but one of them (day 6) has already been scheduled once. The code is reduced to one possibility, day 0, so that is what the TFT flag schedule randomization algorithm chooses. The update results in the following collections:

Color 2

Group 1 = [6]
Group 2 = [1,2,4,5,6]
Schedule = [0,3]

Used Days = [[0,1], [2,1], [3,1], [6,1]]

Choose a third day for TFT Flag 1. The TFT flag schedule randomization algorithm can no longer avoid scheduling a collection within two days of another collection; Group 1 is empty. So, the TFT flag schedule randomization algorithm falls back on the full list of available days in Group 2. The TFT flag schedule randomization algorithm has previously scheduled days 0 and 3, so it can choose from [1,4,5] using the PRNG to generate a random number between 0 and 2. Say the PRNG generates a 0 (day 1). The result is:

Color 1

Group 1 = [ ]
Group 2 = [0,3,4,5]
Schedule = [1,2,6]

Used Days = [[0,1], [1,1], [2,1], [3,1], [6,1]]

Choose the final day for TFT Flag 2. There is still one day left in its list of days not within two days of a previously scheduled day. That day happens to have been scheduled previously for TFT Flag 1, but since that satisfies the minimum days requirement, the TFT flag schedule randomization algorithm still chooses it (day 6) rather than picking a day that has not been scheduled, but will not satisfy the minimum requirement. Thus the final schedule is as follows:

Color 1 Schedule=[1,2,6]

Color 2 Schedule=[0,3,6].

III. Example TFT Flags and Example Randomized TFT Flag Schedules

As mentioned above, TFT flags can be of any suitable type, such as colors. In this section, the TFT flags are simply referred to as “colors” and similar terms. However, sight should not be lost on the fact that each color is a TFT flag and have all of the functionalities of and uses described herein for TFT flags.

FIG. 2 illustrates an example assignment of colors and example corresponding testing frequencies to three groups 200(1) to 200(3) of monitored individuals. In this example, group 200(1) has monitored individuals assigned to one or the other of two colors, here, green (“G”) and tan (“T”), each of which corresponds to a weekly scheduling period 204. Each of these green and tan TFT flags may have the same testing frequency or differing testing frequencies, such as 2 times per week or 3 times per week, among others. Group 200(2) in this example also has monitored individuals assigned to the weekly green and tan TFT flags, but it also has monitored individuals assigned to one or the other of blue (“B”) and pink (“P”) TFT flags. In this example, each of the blue and pink TFT flags has a monthly scheduling period 208. Each of these blue and pink TFT flags may have the same testing frequency or differing testing frequencies, such as 1 times per month or 3 times per month, among others. Group 200(3) in this example also has monitored individuals assigned to the weekly green and tan TFT flags, but it also has at least one monitored individual assigned to a red (“R”) TFT flag, which has a yearly scheduling period 212. The testing frequency of the red TFT flag may be any suitable frequency, such as 5 times a year, 10 times a year, or 16 times a year, among others, as determined by the appropriate authority, such as a medical professional. Group 200(3) does not have any monitored individuals assigned to a monthly frequency. As seen in this example, the weekly period 204 runs from Sunday to Saturday, the monthly period 208 runs from the first day of the month to the last day of the month, and the yearly period 212 runs from January 1^{st} through December 31^{st}.

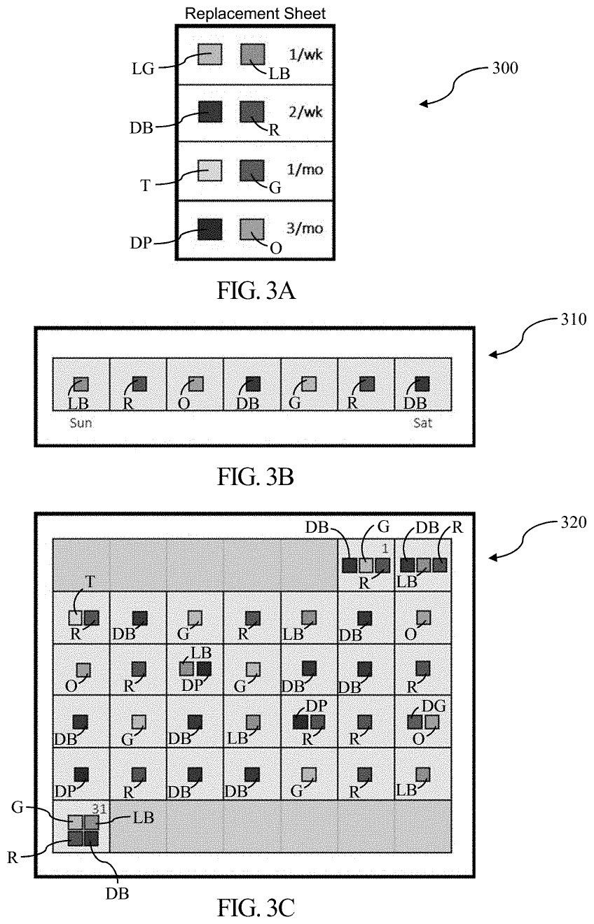

FIG. 3A shows an example testing frequency/color assignment chart 300 for frequencies/colors that an authority, such as a medical professional, can assign to a monitored individual, such as any monitored individual or a monitored individual within a particular group, among others. In this example, the testing frequency/color assignment chart 300 has four differing testing frequencies, namely, once per week, twice per week, once per month, and three times a month, and each testing frequency has two assigned colors. As discussed above, a single frequency may have more than one color assigned to it for any of a variety of reasons. In this example, the once-per-week testing frequency is assigned light green (“LG”) and light blue (“LB”), the twice-per-week testing frequency is assigned dark blue (“DB”) and red (“R”), the once-per-month testing frequency is assigned tan (“T”) and dark green (“DG”), and the three-times-per-month testing frequency is assigned dark purple (“DP”) and orange (“O”).

FIG. 3B shows a week 310 of a randomized TFT flag schedule as randomly scheduled using the frequencies and TFT flag colors of chart 300 of FIG. 3A and a suitable TFT flag schedule randomization algorithm, such as the TFT flag schedule randomization algorithm 132SA of FIG. 1B, which may be the TFT flag schedule randomization algorithm described above in section II. In this example, the TFT flag schedule randomization algorithm has scheduled one TFT flag color scheduled each of the seven days of the week as shown. It is noted that the view illustrated in FIG. 3B may be a view available to a monitoring user via a suitable interface of an RS&T system, such as RS&T system 100 of FIGS. 1A and 1B. As discussed below, when a monitored individual checks an ART notification UI, such as either of ART notification UIs 124(1) and 124(2) of FIG. 1B, they will see (or hear) the TFT flag color scheduled for that day. If that TFT flag color is theirs, then they are due to be tested at a suitable testing location, such as one of testing locations 116(1) to 116(N) of FIG. 1A.

FIG. 3C shows a month 320 of a randomized TFT flag schedule as randomly scheduled using the frequencies and TFT flag colors of chart 300 of FIG. 3A and a suitable TFT flag schedule randomization algorithm, such as the TFT flag schedule randomization algorithm 132SA of FIG. 1B, which may be the TFT flag schedule randomization algorithm described above in section II. In this example, the TFT flag schedule randomization algorithm has scheduled at least one TFT flag color each day of the month, with some days having two TFT flag colors scheduled and some days having three TFT flag colors scheduled. It is noted that the view illustrated in FIG. 3C may be a view available to a monitoring user via a suitable interface of an RS&T system, such as RS&T system 100 of FIGS. 1A and 1B. As discussed below, when a monitored individual checks an ART notification UI, such as either of ART notification UIs 124(1) and 124(2) of FIG. 1B, they will see (or hear) the TFT flag color scheduled for that day. If that TFT flag color is theirs, then they are due to be tested at a suitable testing location, such as one of testing locations 116(1) to 116(N) of FIG. 1A.

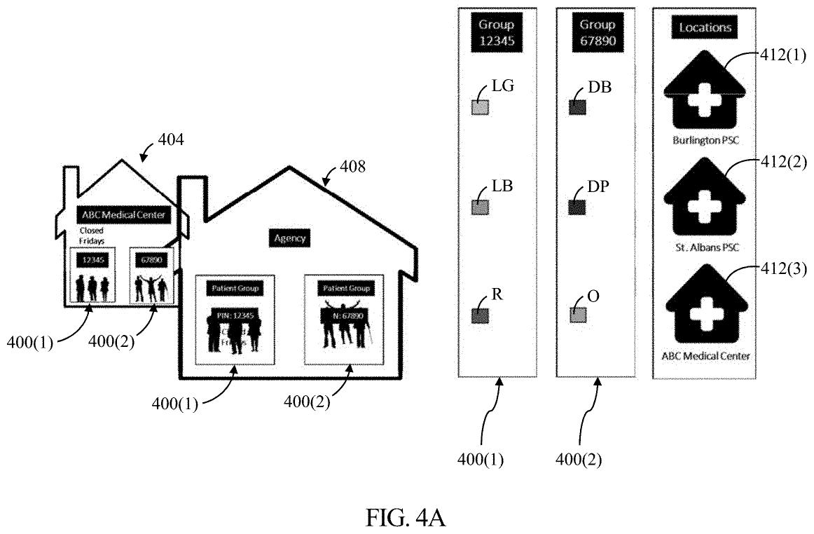

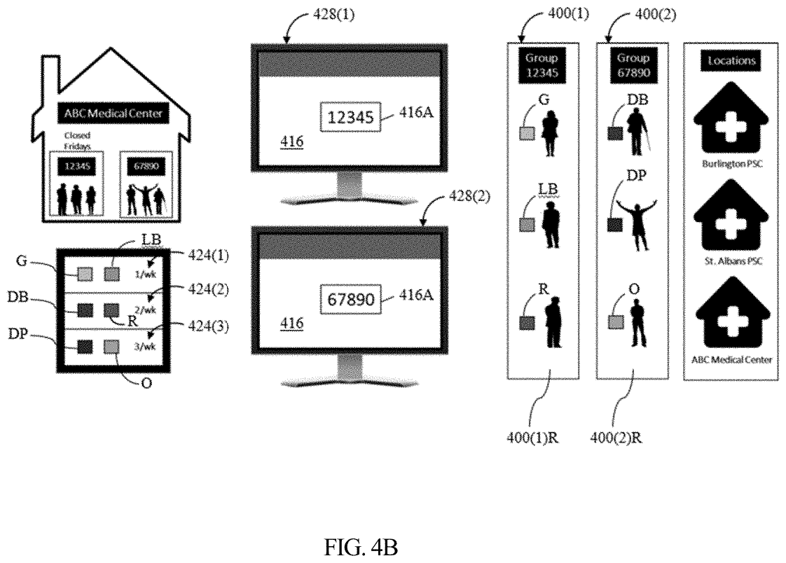

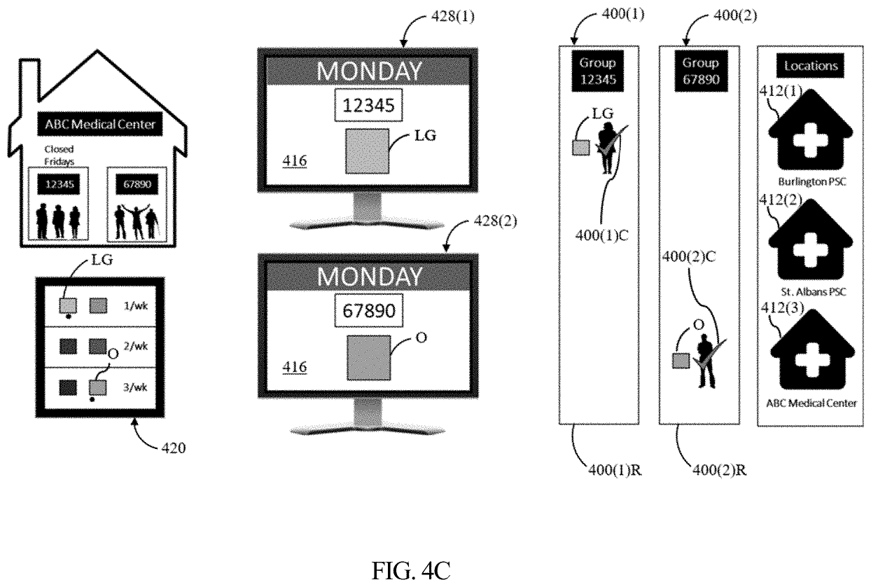

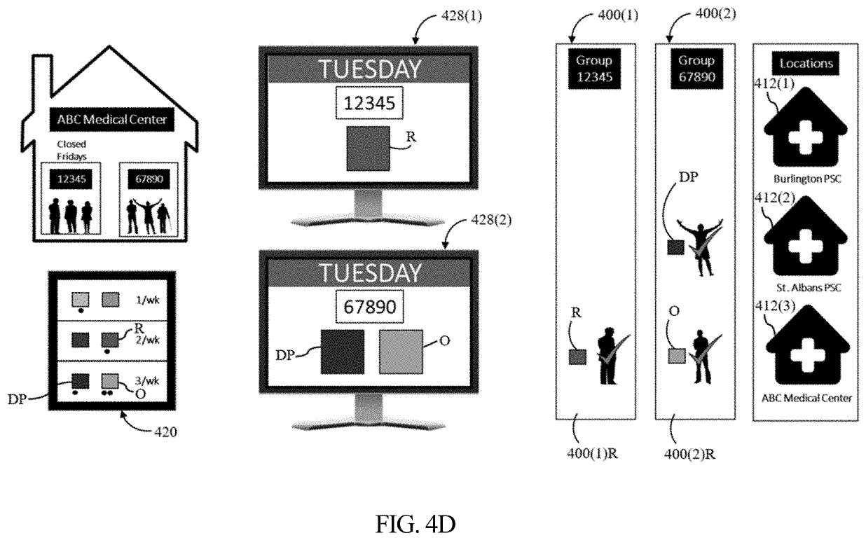

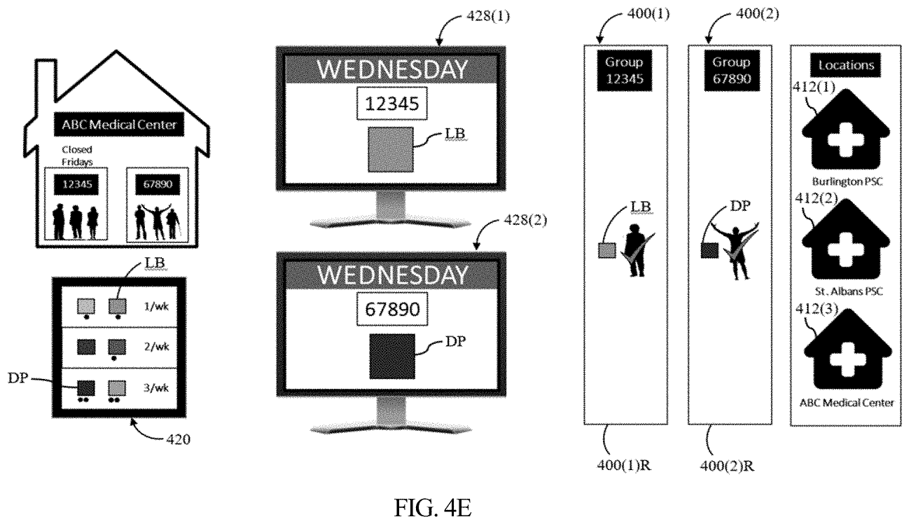

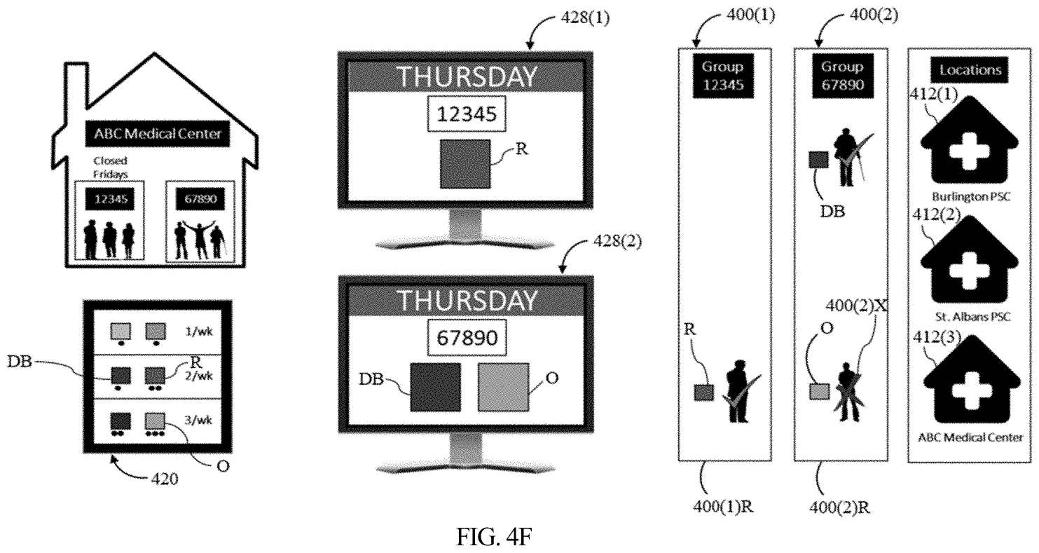

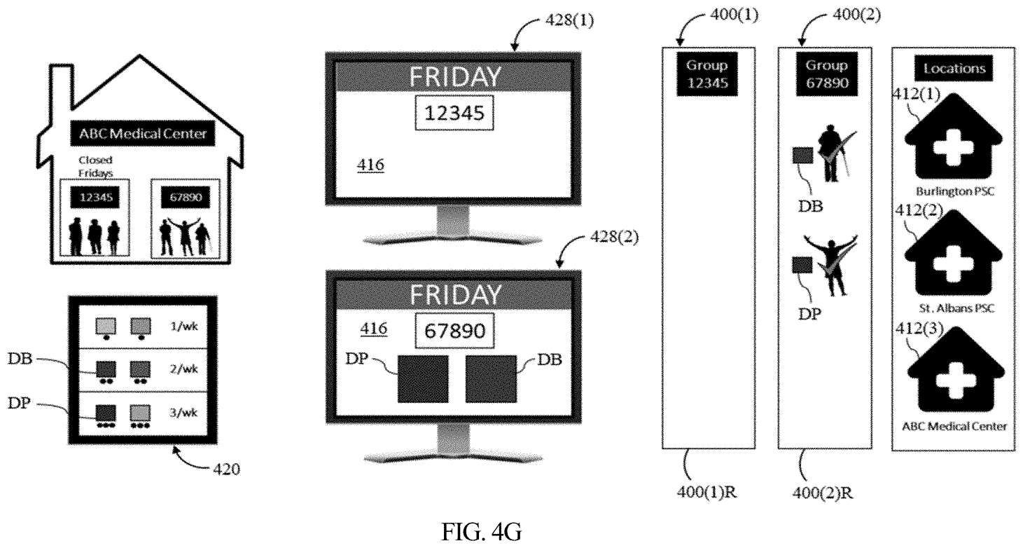

FIGS. 4A to 4G illustrate an example randomized testing scenario for two groups 400(1) and 400(2) of monitored individuals (not labeled, but silhouetted) that are being monitored by a monitoring organization 404, which in this example subscribes to RS&T services provided by a monitoring agency 408. In this example, each of the groups 400(1) and 400(2) has access to three testing locations 412(1) to 412(3), which may be associated or affiliated with one, the other, or both of the monitoring organization 404 and the monitoring agency 408. As those skilled in the art will appreciate, the monitoring organization 404 may be, for example, one of the monitoring organizations 108(1) to 108(N) of the SaaS deployment of FIG. 1A, the monitoring agency 408 may be one of the monitoring agencies 112(1) to 112(N) of the SaaS deployment of FIG. 1A, and each testing location 412(1) to 412(3) may be one of the testing locations 116(1) to 116(N) of the SaaS deployment of FIG. 1A. In this connection, monitoring agency 408 utilizes RS&T system 100 of FIGS. 1A and 1B. In this example, each of the two groups 400(1) and 400(2) has a corresponding PIN that the monitored individuals of that group use to access a suitable ART notification UI 416 (FIG. 4B), which in the example may be web-based ART notification UI 124(1) of FIGS. 1A and 1B. In the example shown, the monitored individuals of group 400(1) have three assigned TFT flag colors, namely, light green (“LG”), light blue (“LB”), and red (“R”), and the monitored individuals of group 400(2) also have three assigned TFT flag colors, namely, dark blue (“DB”), dark purple (“DP”), and orange (“0”). Each group 400(1) and 400(2) may have more or fewer assigned TFT colors and/or differing numbers of assigned TFT flag colors.

FIG. 4B includes a chart 420 that shows three testing frequencies 424(1) to 424(3) (once per week 424(1), twice per week 424(2), and three times per week 424(3)) and the corresponding assigned TFT flag colors. FIG. 4B also includes group regions 400(1)R and 400(2)R that depict the TFT flag color assignments to the monitored individuals (silhouetted) within the corresponding groups 400(1) and 400(2). In this example, it is seen that each monitored individual is assigned a unique color. As noted above, FIG. 4B also shows an ART notification UI 416, which is displayed here on two different computer monitors 428(1) and 428(2) corresponding respectively to the two groups 400(1) and 400(2) (this is only for convenience). In this example, the ART notification UI 416 is accessible via a URL and includes a PIN field 416A that each monitored individual within each of the groups 400(1) and 400(2) enters the PIN assigned to their group to access the TFT flag color(s) (or none) of the day, i.e., the TFT flag color(s) (or none) randomly scheduled using a suitable TFT flag schedule randomization algorithm (not shown), which may be TFT flag schedule randomization algorithm 132SA of FIG. 1B and take the form of the TFT flag schedule randomization algorithm described in section II, above. Here, group 400(1) is assigned the PIN “12345”, and group 400(2) is assigned the pin “67890”.

FIG. 4C to 4G show, respectively, examples of what happens on each of Monday, Tuesday, Wednesday, Thursday, and Friday during a particular week. As seen in FIG. 4C, on Monday, each monitored individual in each group uses the ART notification UI 416 to see the TFT flag color(s) (or none) of the day. In FIG. 4C and as displayed on computer monitor 428(1) in response to a monitored user of group 400(1) entering that group's PIN, the ART notification UI 416 shows that the TFT flag color of the day is light green (“LG”). Similarly and as displayed on computer monitor 428(2) in response to a monitored user of group 400(2) entering that group's PIN, the ART notification UI 416 shows that the TFT flag color of the day is orange (“O”). For the sake of illustration, chart 420 shows each of the light green and orange TFT flags as having a dot (not labeled) beneath it that indicates that it is a TFT flag color of the day. Also for the sake of illustration, FIG. 4C also shows each group region 400(1)R and 400(2)R containing the TFT color of the day and the corresponding monitored individual (silhouetted). In this example, a green check mark 400(1)C and 400(2)C appears overlaid onto the corresponding silhouette to indicate that that monitored individual appeared at one of the testing locations 412(1) to 412(3) and got tested.

As can be readily seen in FIGS. 4D to 4G, the daily routine continues for the rest of the week, with chart 420 tracking the TFT colors called both that day and cumulatively from the beginning of the week (Monday, FIG. 4C), with each computer monitor 428(1) and 428(2) displaying the TFT flag color(s) (or none) of the day for the corresponding group 400(1), 400(2), and with each group region 400(1)R and 400(2)R showing the called TFT flag color(s) (or none) of the day, the corresponding monitored individual, and an indicator (e.g., green check mark or red “X”) indicating whether or not the corresponding monitored individual showed up at a testing location 412(1) to 412(3) and got tested. As can be seen comparing chart 420 across the multiple views of FIGS. 4C to 4G, throughout the week the TFT flag schedule randomization algorithm has scheduled the various TFT flag colors so that each TFT flag color is called the proper number of times that week. See, for example, FIG. 4G (corresponding to Friday), in which chart 420 shows, by way of the number of dots below each color, that RS&T system called each of the once-a-week frequency colors one time in the full week, called each of the twice-a-week frequency colors two times in the full week, and called each of the three-times-a-week frequency colors three times in the full week.

As can also be seen in FIG. 4G, the monitored individuals in group 400(1) are not permitted to be tested on Fridays. Consequently, in FIG. 4G the ART notification UI 416 on the computer monitor 428(1) shows that there is no TFT flag color of the day and that group region 400(1)R is empty. Another difference among FIGS. 4C to 4G is in FIG. 4F, where one of the monitored individuals in group 400(2) failed to show up for a test. This is indicated in group region 400(2)R by the red “X” 400(2)X over the corresponding silhouette.

IV. Example RS&T System and Associated User Interfaces

This section addresses an example RS&T system of the present disclosure, such as the RS&T system 100 of FIGS. 1A and 1B, and some example UIs (mostly GUIs) for such an RS&T system. Accordingly, most of the GUIs addressed in this section are served by one or more servers as webpages, though as noted above, other forms of GUIs can be substituted in different system architectures. Those skilled in the art will readily understand how the example UIs can be implemented in an RS&T system, such as the SaaS-type RS&T system of FIGS. 1A and 1B or a web-based enterprise-type system.

In addition, it is noted that the TFT flags in this example are differing colors. However, as noted above, TFT flags need not necessarily be colors. Those skilled in the art will readily understand how to implement an RS&T system incorporating principles of the present disclosure using TFT flags other than colors. It is also noted that this example is in the context of monitoring “patients” (i.e., monitored individuals) in the context of drug-use rehabilitation. However, those skilled in the art will readily understand that this specific instantiation of the RS&T system can be modified for other types of monitored individuals and deployment contexts and that the necessary modifications can be made by those of ordinary skill in the art.

IV.A Introduction; Administrator Tools; Agency Management

The example RS&T system of this section includes a software application designed to make patient engagement easier, more effective, and better suited to needs of monitoring users, such as treatment providers, hospital administrators, and laboratorians. By reducing the stigma of drug testing in prevention, rehabilitation, and treatment, a goal of this example RS&T system is to ensure that patient treatment protocols are communicated easily, safely, and clearly to both monitoring-user colleagues and patients. The example RS&T system provides patients with an assigned color associated with a specific testing frequency (here, from 3/wk to 1/yr), then randomizes those colors within a defined schedule. Patients are given a number of ways to access their colors including by web, phone, SMS, or e-mail. When their color is chosen, the patient knows to visit a testing location to provide a sample (e.g., urine, blood, etc.). From there, the RS&T system takes over. Patients are marked for attendance so that monitoring users can evaluate trends and adherence to treatment protocols without having to worry about the logistics of scheduling random drug tests.

In this example, the RS&T system is a web-based enterprise-type instantiation, with the various features and functionalities performed and/or provided by one or more web servers (not shown) and accessible by users and patients via a suitable web browser. All GUIs illustrated in this section IV are, therefore, served by the one or more web servers. Like many enterprise-type software systems having a multiuser (e.g., multi-agency) architecture, the RS&T system includes a number of GUIs that provide at least the following functionalities: login credential setup and management; user-profile management; user-notification preferences; two-factor authentication management; and RS&T-system-provider-level administrative controls, including, but not limited to agency management, testing-location management, patient searching, system-provider management, and troubleshooting, among others. These functionalities will be familiar to those skilled in the art and, therefore, need not be illustrated for a skilled artisan to practice the RS&T system to its fullest scope.

IV.B Organization Management

IV.B.1 Profile

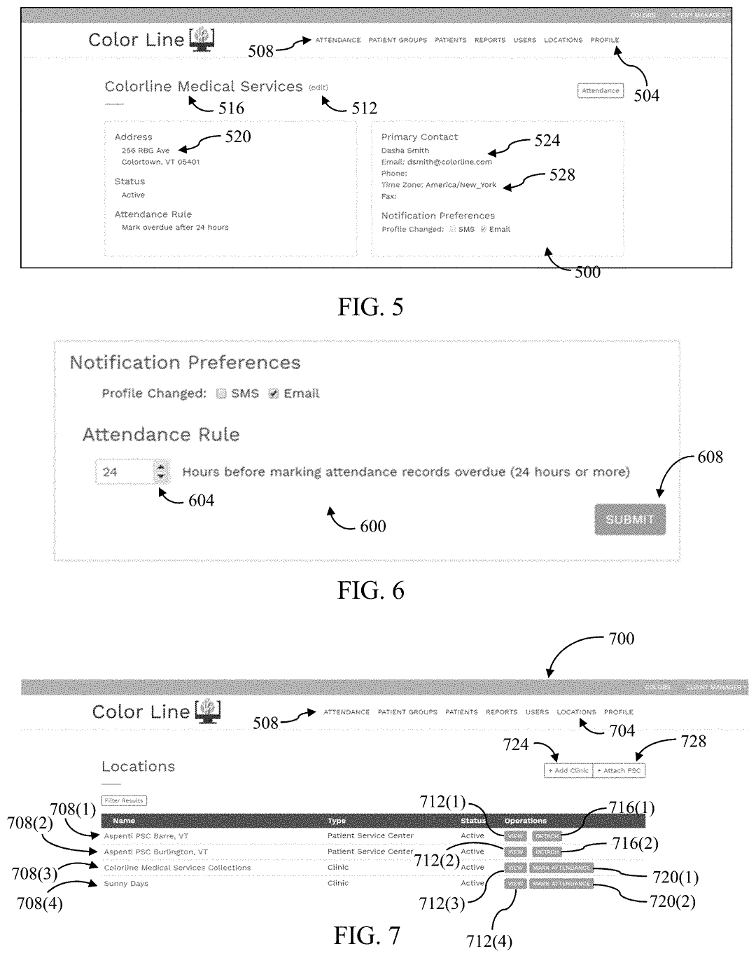

A monitoring organization's profile is the highest tier of organization within the RS&T system. All patients created or uploaded for the current monitoring organization will live within the monitoring organization's profile, and rules applied to the profile will affect all patients of the current monitoring organization. Profiles store information about the organization's main address, attendance rules, and a primary contact (e.g., the primary administrator for the organization). The Profile page 500 of FIG. 5 in this example can be found by clicking a [PROFILE] button 504 on the navigation bar 508.

IV.B.2 Edit Profile

To edit the information stored in the profile, click the [(edit)] button 512 next to the Organization's name 516 on the profile page 500 to bring up an Edit Profile form (not shown). The settings for an organization's name 516, address 520, and primary contact 524 can be changed. When naming the organization's profile, it may be desirable to bear in mind that it should be named broadly enough to encompass all patient groups, providers, and locations being monitored.

IV.B.3 Primary Contact Notifications

The primary contact 524 on the organization's profile can include an e-mail and cell phone number. If populated, the notification preferences 528 can be sent to the contact when changes are made to the profile.

IV.B.4 Attendance Rules

An attendance rule 600 (FIG. 6) is a basis for marking patient attendance. Here, the attendance rule 600 is the amount of time allowed to pass after a color is called before a patient will be marked as “late” or “overdue”. In this example, this amount of time can be raised or lowered in 24-hour increments from one (1) to seven (7) days using the increase-decrease selection tool 604. The value of the attendance rule 600 is settable at the discretion of a professional. Once the user has set a value for the attendance rule 600, the user selects the [SUBMIT] button 608.

For example, if the attendance rule 600 is set to 24 hours and the color “Blue” is called, patients assigned to the color “Blue” will have 24 hours to visit a testing location and be marked as present. If a patient fails to show up, they will be marked as “Overdue”. If they show up after the attendance rule 600 has expired, they will be marked as “Late” unless someone having proper authority (e.g., a healthcare provider) excuses them. If they do not show up at all until “Blue” is called again, they will be marked as “Missed” for the previous collection. As discussed below, each patient can have an individual grace period that can be added to this attendance rule 600, but this attendance rule keeps the minimum amount of time that may pass before an outcome is changed for a patient.

IV.C Testing Locations

IV.C.1 Clinics and Patient Service Centers

Testing locations are specimen collection centers that a patient can visit. In this instantiation, testing locations can be owned and managed by the enterprise hosting the RS&T system or by any of the organizations using it to engage patients. The former, i.e., locations owned and managed by the hosting enterprise, are called “patient service centers” (PSCs) in this instantiation. The latter, i.e., locations owned and managed by the organization or providers using it, are called “clinics” in this instantiation. Both types of locations have the same functionality but differ in how they manage patients. While the hosting enterprise can view many patients from different organizations, a particular organization will view only patients belonging to that organization and its patient groups. Users belonging to a particular organization can mark attendance at any of that organization's clinic locations, but only hosting enterprises can mark attendance at one of their owned PSC locations. To view a testing-locations page 700 (FIG. 7), a user can click the [LOCATIONS] button 704 in the top navigation bar 508. Once there, the user can view a particular location, here, any one of locations 708(1) to 708(4) by clicking the name or the corresponding [VIEW] button 712(1) to 712(4).

As can be readily seen in FIG. 7, testing locations 708(1) and 708(2) are PSCs, whereas testing locations 708(3) and 708(4) are clinics, and these two types of testing locations have two differing operations that a user can select to take via corresponding respective [DETACH] buttons 716(1) and 716(2) and [MARK ATTENDANCE] buttons 720(1) and 720(2). Since the hosting enterprise is in direct control of its PSCs, its users have the option of detaching a PSC, such as either of testing locations 708(1) and 708(2). However, the hosting enterprise will typically not have such direct control over clinics, such as testing locations 708(3) and 708(4) that are typically under the direct control of one or more monitoring organizations and, as such, providing the hosting enterprise with detaching functionality is not appropriate. However, in this example the testing-locations page 700 provides each of the clinic testing locations 708(3) and 708(4) with the corresponding [MARK ATTENDANCE] button 720(1) and 720(2) that allows a hosting-enterprise user to access attendance functionalities, which are described below.

In this example, testing-locations page 700 also includes a [+ Add Clinic] button 724 and a [+ Attach PSC] button 728 that allow a hosting-enterprise user to, respectively, add a new clinic to the testing locations available in the RS&T system and attached a new PSC to the testing locations available in the RS&T system.

IV.C.2 Adding a Clinic

To add a new clinic, the user selects the [+ Add Clinic] button 724 on the testing-locations page 700. Although not shown, information to be entered for the new clinic includes a name, address, and primary contact. This is similar to the information shown on an organization profile page, such as profile page 500 of FIG. 5. Also similar to the profile page 500 of FIG. 5, a user can set notification preferences (see the notification preferences 528 of FIG. 5) for the primary contact of the new clinic. Not shown is that the RS&T system can store information regarding how a collection is requisitioned and transmitted to a lab. In one example, there are selections for submitting a requisition through an enterprise-run lab, an interfaced electronic medical record (EMR) system, or a Manual paper requisition.

IV.C.3 Attaching a PSC

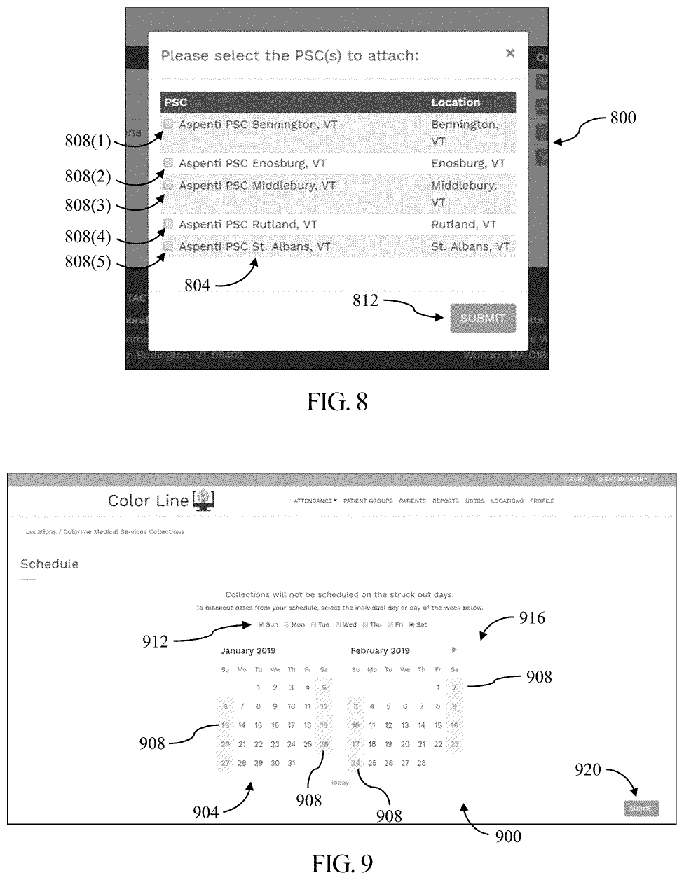

FIG. 8 illustrates a popup window 800 that the RS&T system displays in response to a user selecting the [+ Attach PSC] button 728 on the testing-locations page 700 of FIG. 7. In this example, the popup window 800 includes a list 804 of enterprise-controlled PSCs, with each entry having a corresponding check box 808(1) to 808(5) that the user can select to add the corresponding PSC to the active PSCs. Once the user has selected the desired PSC(s), they select the [SUBMIT] button 812 to effect the addition of the selected PSC(s) to the active PSCs.

IV.C.4 Testing-Location Schedules

One of the most important functions of a testing location is to keep a schedule. In this example of an RS&T system, testing-location schedules can be modified to accommodate work hours and can be updated as late as the night before a closure. To access the schedule of an existing clinic, a user can navigate to a clinic page (not shown), or PSC page (not shown) if the user is an enterprise user, and select a [Schedule] button (not shown). In response to this selection, the RS&T system displays a schedule page, such as the schedule page 900 of FIG. 9, containing a calendar 904 that shows the days that are available for testing. In the example of FIG. 9, the days that are crossed-out by hatching 908 are not eligible for colors to be called on those days.

In this example, recurring days and individual days can be selected and thereby be “turned-off” such that the RS&T system does not call colors for those days. This can be useful, for example, for managing irregular work weeks, holidays, inclement weather, or emergency closures. To turn a day “off” from displaying colors, the user can simply click the corresponding day on the calendar 904. To turn off every recurrence of a specific day, click a corresponding one of checkboxes 912 indicating the day that should be turned off. The schedule calendar 900 can be modified for an entire calendar year, with arrows, such as arrow 916, provided so that the user can scroll through future months and then back to a previous month, as desired. In this example, at the restart of the new year, the RS&T system will repeat any previously selected recurring days and reset individual days. Once the schedule has been set up, the user selects a [SUBMIT] button 920 to save the changes.

IV.D Patient Groups

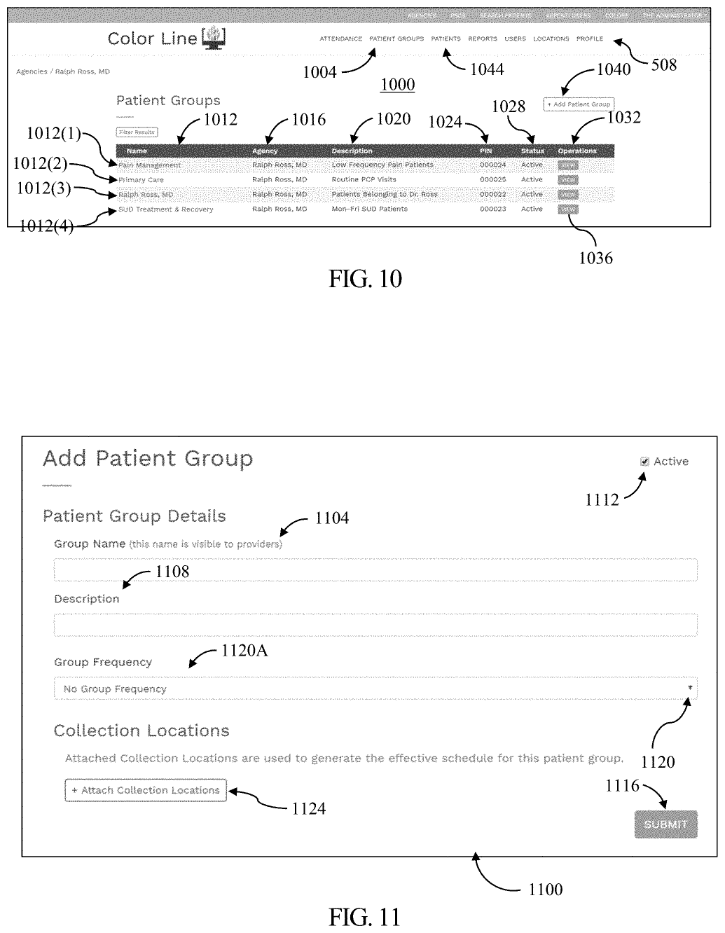

FIG. 10 illustrates an example patient-group page 1000 that a user may access, for example, by selecting a [PATIENT GROUPS] button on a navigation bar of certain pages of the RS&T system, such as [PATIENT GROUPS] button 1004 on the navigation bar 508 in FIG. 10 that is present on many of the pages that the RS&T system serves to a user. Patient groups allow a user to categorize treatment plans and schedules into separate patient pools. Each patient group has its own schedule, can consider the schedules of different locations, and utilizes a TFT flag schedule randomization algorithm to display colors according to the group setup and color frequencies of the patients within that patient group.

For example, a patient group can be set up to only display colors on Monday through Friday, on only Tuesdays and Thursdays, or at any time. A patient group can be set up to only display colors when a collection location (a clinic or a PSC) is open, or to only display colors when multiple collection centers are open. A patient group can be set up to use a group frequency, where all patients within that patient group are assigned to the same testing frequency. A patient group can alternatively be set up to group patients with a variety of testing frequencies within the same schedule. How patient groups are set up will determine when patients see that their color has been picked. Each patient group has a PIN that is shared with all patients inside the group, and PINs can be used to display colors via phone or web if the patient does not receive SMS or email notifications.

In this example, patient-groups page 1000 has a group name column 1012, an affiliated agency column 1016, a group description column 1020, a PIN column 1024, a status column 1028, and an operations column 1032. To view a patient group, a user can either select that patient group's hyperlinked name 1012(1) to 1012(4) or a [VIEW] button 1036 from the patient groups page 1000.

IV.D.1 Adding a Patient Group

A user can elect to add a new patent group by selecting an [+ Add Patient Group] button 1040 on the patient-groups page 1000. In response to this selection, the RS&T system may display the add-patient-group page 1100 of FIG. 11. In this example, the add-patient-group page 1100 includes: a group name field 1104 where the user can input a name for the new patient group, a group description field 1108 where the user can input a brief description of the new patient group, an [Active] checkbox 1112 that the user can select to make the new patient group an active group, and a [SUBMIT] button 1116 that the user selects when done entering the relevant information for the new patient group.

IV.D.1.i Group Frequencies

While best practice recommends that each patient be assigned an individual frequency according to their treatment plan, some organizations may benefit from utilizing the same frequency of testing for all member patients of the patient group. This could be, for example, for athletic testing, employer drug testing, or other non-substance-use-based treatment applications. In this connection, the example add-patient-group page 1100 of FIG. 11 includes a group-frequency selector, here, a drop-down-menu selector accessible via a down-arrowhead 1120. The group-frequency selector allows the user to select either a “No Group Frequency” selection 1120A, which causes the RS&T system to use the frequencies assigned to individual member patients of the new patient group, or any of one or more frequency selections (not shown) that, when selected, causes the RS&T system to use the selected frequency for all patient members of the new patient group.

IV.D.1.ii Attaching Patient Groups to Collection Locations

A user can attach a new patient group to one or more collection locations that are already created or associated with the organization. See section IV.C, above. A user can do this by selecting a [+ Attach Collections Locations] button 1124 on the add-patient-group page 1100 of FIG. 11. In this example, this selection causes the RS&T system to display a testing-location-selection page (not shown) that display a list of all active testing (collection) locations available for the new patient group. When a group is attached to a collection location, the schedules of the location and the new patient group are combined to create a schedule that blocks days according to each of the collection-location schedule(s) and the patient-group schedule. This is called an “effective schedule” and is discussed below in section IV.D.2.ii.

In this example, attaching a patient group to a testing location affects how patient members of that patient group receive their daily colors. Consequently, if a patient visits multiple collection locations, and only one is attached to their patient group, that collection location will command the schedule of all patients within the group, including those who primarily visit another location.

IV.D.1.iii Patient Group PINs

In this example, the RS&T system automatically generates a unique five-digit PIN for each new patient group. See the PIN column 1024 of the patient-groups page 1000 of FIG. 10. As discussed above and below, a patient member of a particular patient group can use that patient group's PIN to access the colors of the day via a suitable ART notification UI, examples of which for this example of RS&T system are described below in section IV.G. The PIN is the same for all patients within the corresponding patient group.

IV.D.2 Patient-Group Schedules

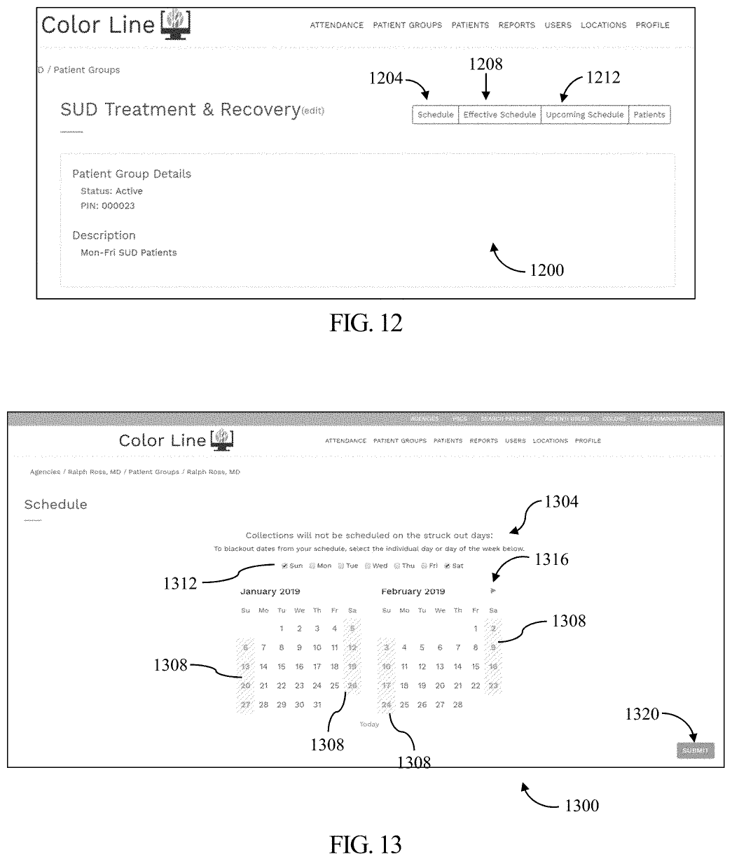

In this example, each patient group has three types of schedules found on a corresponding patient-group profile page: the group schedule, the effective schedule, and the upcoming schedule. While the group schedule is vital to the patient group, the effective schedule is also important. This section describes how to use all three types to ensure flexibility, effectiveness and ease when configuring the patient group. An example patient-group page 1200 is shown in FIG. 12. In the example of FIG. 12, the patient-group page 1200 includes a [Schedule] button 1204 that allows a user to access the corresponding patient-group schedule, here patient-group schedule 1300 of FIG. 13, an [Effective Schedule] button 1208 that allows the user to access the effective schedule (not shown) for the current patient group, and a [Upcoming Schedule] button 1212 that allows the user to access the upcoming schedule for the current patient group, such as the upcoming schedule 1400 of FIG. 14. Each of the group schedule, effective schedule, and upcoming schedule is described immediately below.

IV.D.2.i Group Schedule

In this example, the patient-group schedule 1300 of FIG. 13 is managed using the same process as for a testing-location schedule as discussed above in section IV.C.4. That is, days on a calendar 1304 crossed-out by hatching 1308 indicate the days that colors are not scheduled. Individual days can be “turned-off,” as can recurrences of specific days of the week. The user can click on an individual day to toggle it on or off and can use the checkboxes 1312 at the top of the calendar 1304 to toggle recurrences of a specific day. The schedule calendar 1304 can be modified for the entire calendar year, and the arrows (such as the arrow 1316) can be used to scroll through future months and back. At the restart of the new year, the RS&T system will repeat any previously selected recurring days and reset individual days. Once the user has set up the current patient group's schedule, they can select the [SUBMIT] button 1320 to save the changes.

IV.D.2.ii Effective Schedules

For each patient group, the effective schedule is a combination, or fusion, of the corresponding patient-group schedule and the schedules of all attached active collection locations. The patent-group's effective schedule is the true schedule that will result in a color palette being displayed or not on a patient-facing “colors” page (see second IV.G, below) and on the current patient group's upcoming schedule, such as illustrated in the example upcoming-schedule page 1400 shown in FIG. 14. For example, if the patient-group schedule is “open” Monday through Friday but is fused with a testing-location schedule of a collection location that is closed on Wednesdays, then the effective schedule will show Monday, Tuesday, Thursday, and Friday as the available days for collections.

Because an effective schedule is a combination of other schedules, namely, a patient-group schedule and a testing-location schedule, it cannot be edited. To revise an effective schedule, changes must be made to the corresponding patient-group schedule and/or to the testing-location schedule.

IV.D.2.iii Upcoming Schedules

As mentioned above, FIG. 14 shows the example upcoming-schedule page 1400, which displays the colors that will be called for the next four weeks, one week at a time. As seen on the upcoming-schedule page 1400, the displayed week 1404 is week 2 out of the four weeks, as indicated by the highlighted [Week 2] button 1408(2) of the four [Week] buttons 1408(1) to 1408(4). Because upcoming schedules can change due to new-patient assignments and modifications to patient-group schedules and/or testing-location schedules, in the present example the RS&T system determines weekly frequencies for only two weeks while it displays monthly and yearly colors through all four weeks. This upcoming-schedule page is not editable, but it can provide valuable information about upcoming color palettes to monitoring users, such as healthcare providers or managers.

IV.D.3 Patient-Group Patients

IV.D.3.i Viewing Patients with a Patient Group

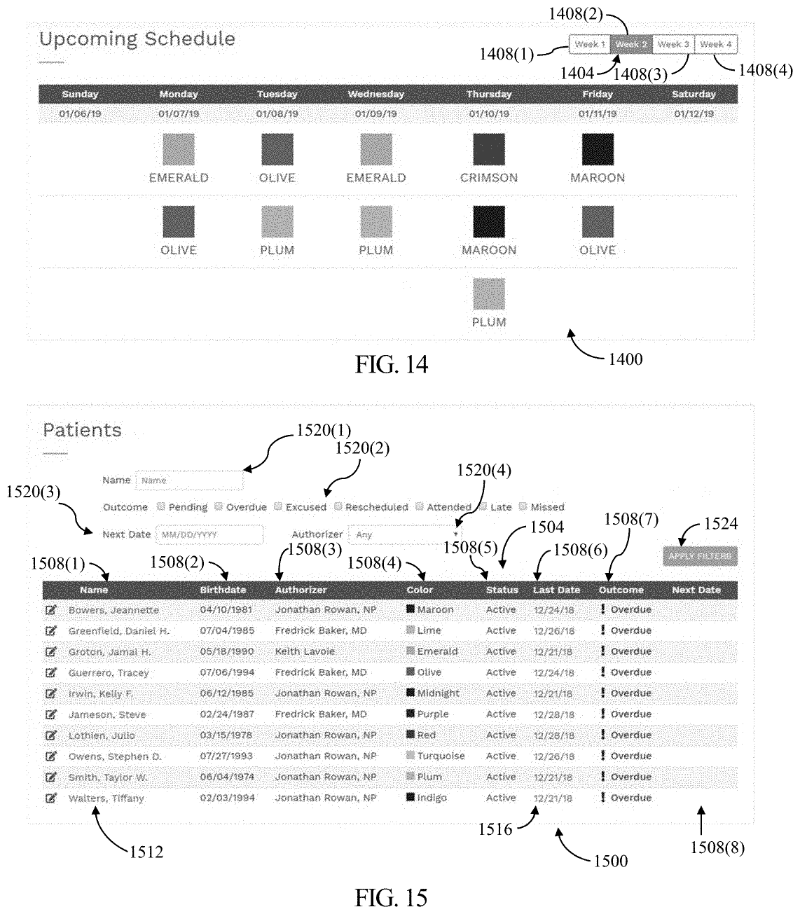

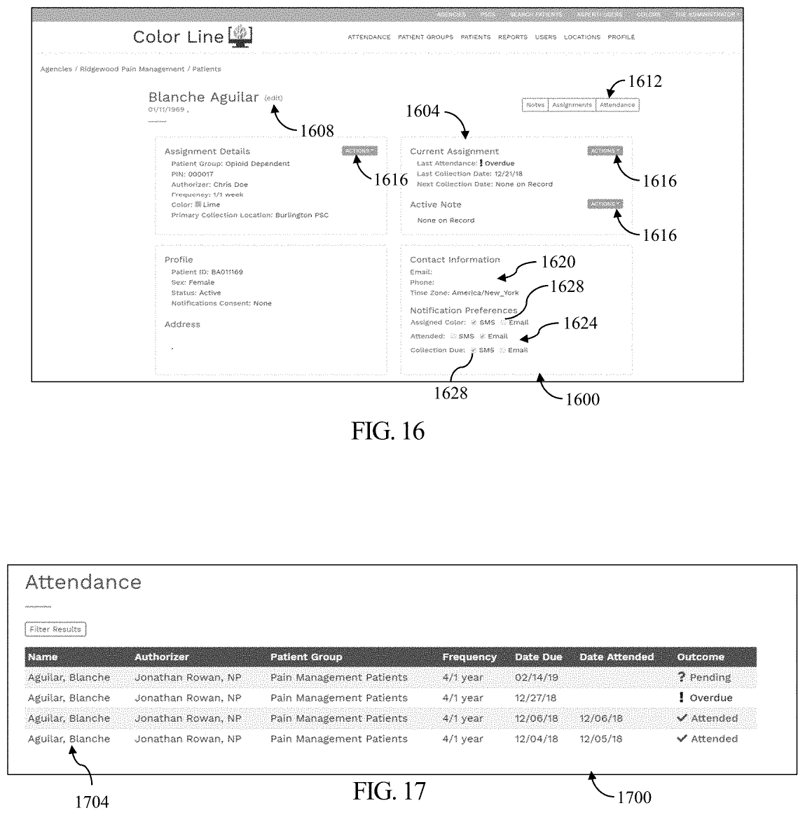

To view all patients associated with a patient group, a user can select a [PATIENTS] button on a patient-group page, such as the [PATIENTS] button 1044 on the navigation bar 508 on the patient-group page 1000 of FIG. 10 or other page. In response to such selection, the RS&T system may display a patients page, such as the patients page 1500 of FIG. 15, which includes a patients table 1504 that includes a name column 1508(1), a date of birth column 1508(2), an authorizer column 1508(3), an assigned color column 1508(4), and a status column 1508(5), as well as a last-due-date column 1508(6) and an outcome column 1508(7), and a next-predicted-due-date (if scheduled) column 1508(8). Clicking on any of the hyperlinked patient names 1512 will cause the RS&T system to navigate to that patient's patient-profile page, such as the patient-profile page 1600 shown in FIG. 16. Clicking on any of the hyperlinked last due dates 1516 on patients page 1500 of FIG. 15 will navigate to an attendance record (not shown) associated with that due date. A user can use a set of filters 1520(1) to 1520(4) to filter this list 1504 by name, outcome, next due date, and authorizer to show a particular subset of patients by setting the desired filter(s) and then selecting an [APPLY FILTERS] button 1524.

IV.D.3.ii Patient Profiles

As mentioned, FIG. 16 illustrates an example patient-profile page 1600. Once a user has created a patient within the RS&T system, that patient's profile can be viewed and edited. In the present example, patient profiles can be accessed from any page—other than the patient-profile page—that shows a patient's name as a hyperlink. Clicking on the hyperlinked name from the Patients page or elsewhere will navigate to that patient's patient-profile. As seen on the example patient-profile page 1600, the patient-profile page displays basic demographic information about the patient, including their name, date of birth, unique patient identifier, gender and status.

If the patient has been given an assignment in the current-assignment region 1604, in this example the patient-profile page 1600 displays their patient group, group PIN, authorizer, testing frequency, color, and primary collection location. The patient-profile page 1600 may also display the patient's latest collection date, the outcome of that visit, and their next collection date as a part of their assignment. A user can edit information on the patient-profile page 1600 by selecting the [(edit)] button 1608.

IV.D.3.iii Attendance History

Referring to FIG. 16, by a user selecting an [Attendance] button 1612 on the patient-profile page 1600 or by selecting a [View Attendance] option (not shown) displayed in a drop-down menu (not shown) activated by an [ACTIONS] button 1616, the RS&T system will display the current patient's complete attendance records, such as in the attendance history 1700 of FIG. 17. This list in the attendance history 1700 will grow as the RS&T system appends new assignment attendance records to old ones, thereby allowing the user to see changes in assignments as they correlate to attendance records. Clicking on any of the individual assignments 1704 in the attendance history 1700 causes the RS&T system to display the complete attendance record for that day, including any notes.

IV.D.3.iv Patient Notification

Referring again to FIG. 16, if a patient has an email and/or phone number listed in the “Contact Information” region 1620 of their patient-profile page 1600, the RS&T system can use this information to provide notifications to that patient, depending on the notification preferences set in the “Notification Preferences” region 1624 of the patient-profile page. In this example, patients can receive three types of notifications:
Assigned Color: The RS&T system sends the patient an SMS text message or email if the patient has a color assignment. The message the patient receives displays the assigned color, PIN number, and a link containing the PIN for their specific Colors page. The patient will only receive the notification once per assignment, but can reuse the link in the message to visit the Colors page daily.
Collection Due: The RS&T system sends the patient an SMS text message or email if the patient has a specific color due. In this example, the patient will receive the message at 8:00 AM in their time zone, as determined by the information on their patient-profile page.
Attended: The RS&T system sends the patient an SMS text message or email after they have attended their collection and been logged for attendance.

Notification preferences can be set at any time, and adjusted as needed. Before a patient begins to receive SMS or email notifications, they must consent to receive them. Once a patient's consent has been received and documented for either SMS messaging, email, or both, a user should check the corresponding consent box(es) 1628 (only two labeled for convenience) in the “Consent for Notifications” region 1624 on the patient-profile page 1600. Until a user has selected a consent box 1624, the RS&T system will not send any notifications regardless of other preferences.

IV.E Patient Assignment

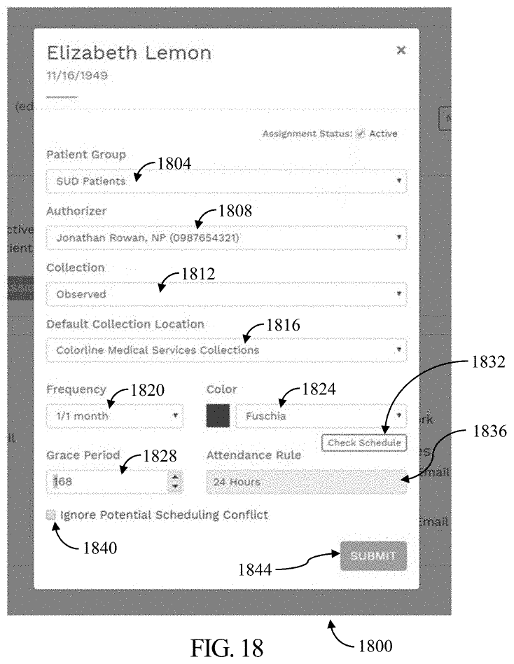

FIG. 18 illustrates an example patient-assignment card 1800 that a user can use to set up a new patient or modify information concerning a patient already existing within the RS&T system. In this example, the patient-assigment card 1800 includes a Patient Group selector 1804, an Authorizer selector 1808, a Collection selector 1812, a Default Collection Location selector 1816, a Frequency selector 1820, a Color selector 1824, and a Grace Period selector 1828. In this example, each of the selectors 1804 to 1828 are of the drop-down menu type.

The organization's patient groups are available as selections via the Patient Group selector 1804. The user chooses one from a drop-down list (not shown) according to the patient's plan. If a patient group utilizing a group frequency is selected, it will prevent this patient from being assigned an individual frequency via the Frequency selector 1820. Otherwise, the user can select the appropriate testing (collection) frequency from a drop-down list (not shown) accessible via the Frequency selector 1820.

The user uses the Authorizer selector 1808 to assign the patient an Authorizer based on users created for the organization with an “Authorizer” role. Upon user selection of the Authorizer selector 1808, the RS&T system displays a drop-down list (not shown) showing all available authorizers. In this embodiment, the selected authorizer will be able to view this patient when logged in to the RS&T system.

The “Collection” selector 1812, when selected, displays options for how to collect the specimen. This is usually whether or not the collection should be observed, unobserved, or “window observed”. A collector at a testing (collection) location can see this selection when marking attendance, allowing them an easy way to be informed of the proper protocols.

The Default Collection Location selector 1816, when selected, provides a drop-down list (not shown) of all collection locations associated with the current organization. The collection location assigned as the default will get the associated patient listed on a roster when the patient is expected to come in based on the randomized scheduling. However, the patient can be found if they show up at another location.

As discussed above, the testing frequency is the number of times per period that the patient's color is selected. In this embodiment, the testing frequency can be any frequency from as low as one time per year to as high as three times per week. Frequencies will be coordinated with group schedules to ensure that the correct frequency is resolved for each period and schedule. It is recommended that patients do not know their test frequency to ensure the integrity of each collection. As noted above, if the current patient is not assigned to a patient group having a group testing frequency, the Frequency selector 1820, when user-selected, will display a drop-down list (not shown) containing all available testing frequencies.

IV.E.1 Color Assignments

For each available frequencies, there can be a number of color options available via the Color selector 1824. As discussed above and below, the assigned color set via the Color selector 1824 is communicated to the patient and displayed on the Colors page as an indicator that the patient must visit a collection location. When multiple colors are available for the selected frequency, there is no difference to which of the available colors is chosen as long as the selected frequency does not change.

The patient-assignment card 1800 includes a [Check Schedule] button 1832 that can assist a user in preventing issues with scheduling new assignment colors that are already in-use within the assigned patient group. Due to inherent risk with randomization, there is the potential for a patient to be assigned a color that has recently been called and may not be called again until the end of the next frequency period. For example, if the color Orange, picked once per month, is already in-use for a patient group, it may be called on the first day of the month. If a new patient is assigned Orange on the second day of the month, they have already missed that month's instance of the color. If the color is not called again until the last day of the second month, the new patient may end up waiting nearly two months for their first collection. To help avoid that problem, the user's selection of the [Check Schedule] button 1832 allows the user to see (not shown) the last and next dates the color will be called. If the color was just recently called or occurs too soon or too far from the date of the assignment, the user can select a different color.

A grace period is an additional time period after the Attendance Rule period. A user can use the Grace Period selector 1828 to give the current patient up to seven additional days, in 24-hour increments, of additional time to visit before their attendance outcome is changed. In this example, the patient-assignment card 1800 also displays the current organization's Attendance Rule 1836 to inform the user of the existing attendance rules before adding a grace period to the assignment.

If a patient is reassigned to a new color in the middle of the week, the RS&T system will attempt to reject color selections if it will result in the patient being called too many times within a period. For example, if a patient is assigned to a twice-per-week frequency, and is called Monday and Tuesday, but then gets reassigned to a three-per-week color on Wednesday, the RS&T system will reject colors that result in that patient being called more than once before that particular week is over. This prevents that patient being called four or more times in a week, despite selections for only two and three collections. In this embodiment, the patient-assignment card 1800 provides an [Ignore Potential Scheduling Conflict] selector 1840 that gives the user the option to ignore this rejection and proceed with the new assignment anyway by selecting this [Ignore Potential Scheduling Conflict] selector. If utilized, it is recommended that the user review the patient's history to ensure no problems arise with medical necessity. When the user is done with entering all of the information for the current patient assignment, the user can select the [SUBMIT] button 1844 to save the patient assignment information. If the patient is configured to receive notifications on assignment, as discussed above the RS&T system will send the patient an SMS text message or email informing them of their assignment.

IV.F Testing (Collection) Attendance

IV.F.1 Patient Rosters

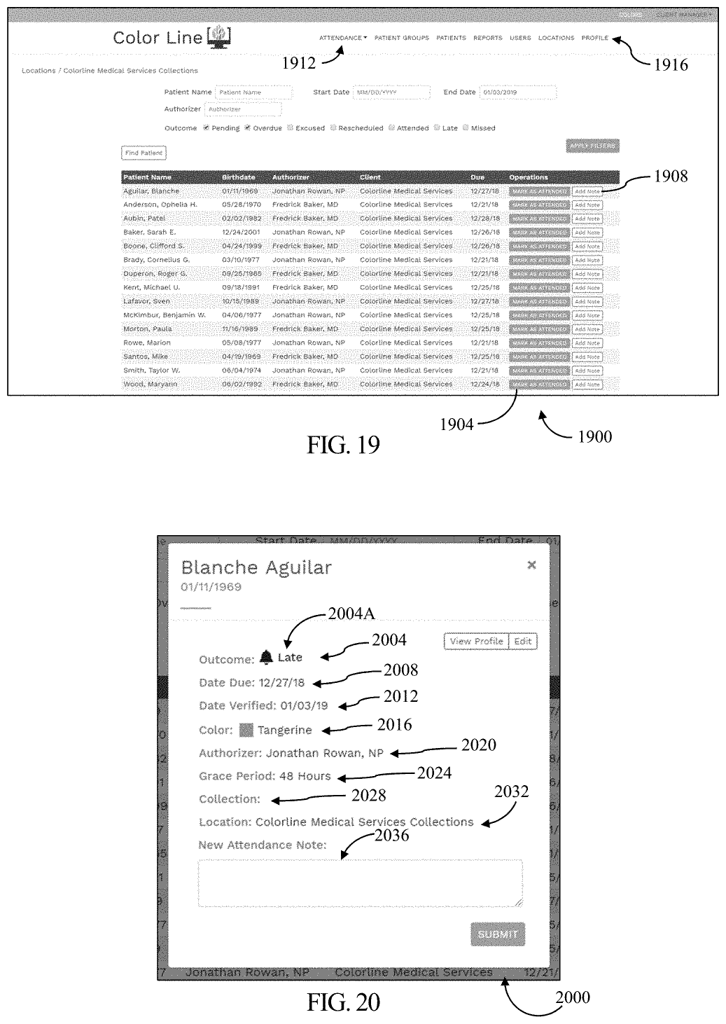

FIG. 19 illustrates an example expected-patients-roster page 1900. When a patient is assigned to a default collection location, the patient is placed on an expected-patients-roster page for collectors, such as expected-patients-roster page 1900.

IV.F.1 Marking Attendance

When the patient arrives for a test, a user, for example, the specimen collector, marks the patient as having attended the collection using a [MARK AS ATTENDED] button 1904 (only one labeled for convenience). When the user selects the [MARK AS ATTENDED] button 1904, the RS&T system displays a patient-attendance card, such as the patient-attendance card 2000 shown in FIG. 20. As seen in FIG. 20, the patient-attendance card 2000 includes an Outcome field 2004, a Due Date field 2008, a Date Verified field 2012, a Color field 2016, an Authorizer field 2020, a Grace Period field 2024, a Collections field 2028, and a Location field 2032.

The outcome field 2004 shows an outcome 2004A that the RS&T system determines by the date due, date verified, attendance rule, and patient grace period. If the date verified supersedes the date due by a factor greater than the patient's grace period, the RS&T system marks the patient as “Late”. If the patient attends within their period, the RS&T system marks the patient as “Attended.” The RS&T system populates the Due Date field 2008 with the date the patient's color was called. The Date Verified field 2012 contains the date that the patient arrived at the collection location for specimen collection. The Color field 2016 displays the color assigned to the patient. The Authorizer field 2020 displays the authorizer or provider assigned to the patient. The Grace Period field 2024 displays the total hours past the due date that the patient may arrive for testing. This is a combination of both the attendance rule and the individual grace period in the patient assignment. The Collections field 2028 contains the type of collection, such as “Observed”, “Not Observed,” and “Window Observed”, or can be blank if not specified. The Location field 2032 shows the patient's default collection location as determined by their patient assignment.

IV.F.2 Reviewing Attendance

In this example, the patient-attendance card 2000 also includes a New Attendance Note field 2036 in which the user can enter attendance notes about the patient. If present, the RS&T system can save and associate the attendance notes with the collection date of the patient. These attendance notes differ from patient notes discussed above in that they are associated with the attendance record for the patient instead of the patient themselves. Both notes can exist concurrently. Notes can be added either by adding text to the attendance card when marking attendance or by selecting an [Add Note] button 1908 (only one labeled for convenience) on the expected-patients-roster page 1900 (FIG. 19). A patient does not need an outcome for the user to add an attendance note, and notes can be reviewed through the attendance history list found within the patient profile as noted above.

IV.F.3 Attendance Reports

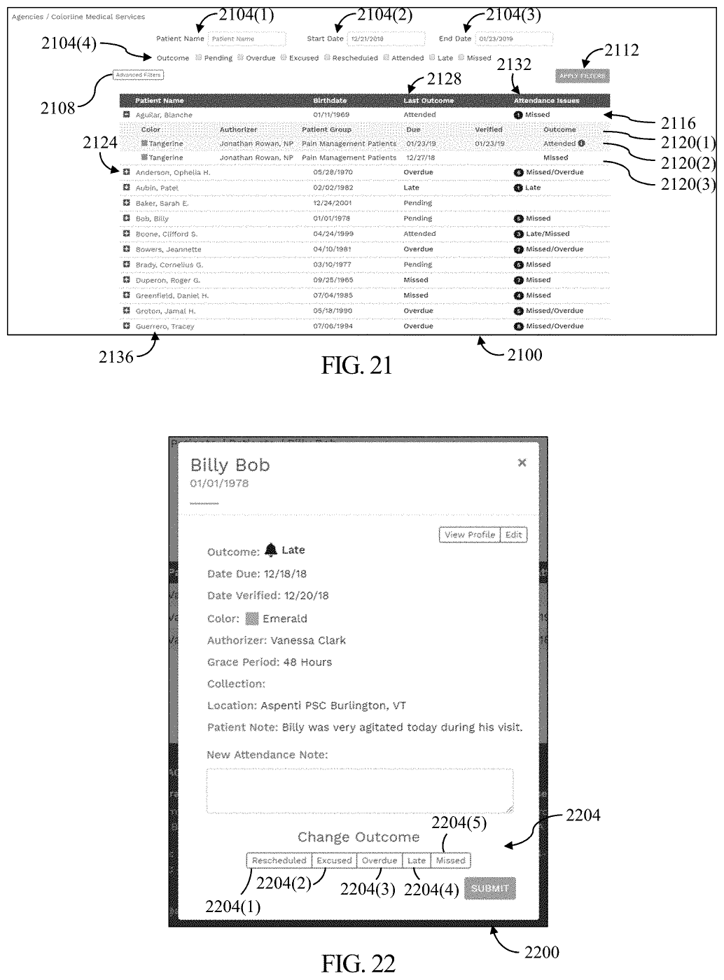

Once a patient has visited a collection location to provide a specimen in response to them learning that their color was called by the RS&T system and collection-location staff has documented that patient's attendance, the RS&T system will track their attendance. To review attendance, a user may navigate to an attendance page, such as the attendance page 2100 of FIG. 21, by selecting an [Attendance] button on the top navigation bar of any of a variety of pages served by the RS&T system, such as the [Attendance] button 1912 on the top-navigation bar 1916 of the expected-patients-roster page 1900 of FIG. 19. In this embodiment, the attendance page 2100 (FIG. 21) is the homepage for users with the authorizer role.

Using corresponding respective ones of filter controls 2104(1) to 2104(4), a user can filter the results presented on the attendance page 2100 by name and date range, or to show specific outcomes. If the user selects an [Advanced Filters] button 2108, in this example the RS&T system displays additional filter settings (not shown) to allow the user to sort by status, authorizer, and/or patient group. Once the user has set the desired filter control(s) 2104(1) to 2104(4), the user may select an [APPLY FILTERS] button 2112 to cause the RS&T system to apply the corresponding filter(s).

In this example and as seen in FIG. 21, the attendance page 2100 displays each patient in a parent row 2116 (only two labeled for convenience), each of which can have a number of child rows 2120(1) and 2120(3) (only three shown). A user can expand any parent row 2116 by selecting the corresponding [+] button 2124 (only one labeled for convenience) to the left of the corresponding parent row 2116. Child rows 2120(1) to 2120(3) show the corresponding patient's assignment details and the outcome of each attendance record associated with that assignment.

IV.F.3.i Attendance Outcomes

When patients are given an assignment, as noted above they are allotted a specific number of days to show up to a collection location as determined by their organization's attendance rule and individual grace period. If a patient visits a collection location inside of their allotted time, they are given an “Attended” outcome. If the patient does not make it to a collection location before their time runs out, they are marked as “Overdue” or “Late”, depending on whether they show up or not. In this embodiment, there are a total of six possible attendance outcomes:
Attended: Patient arrived at a collection location within their allotted time period.
Overdue: Patient has not arrived at a collection location and their time period has run out.
Late: Patient arrived at a collection location after their allotted time period ran out.
Missed: Patient did not visit a collection location after their color was called, and their color has been called again.
Excused: A provider or manager has marked their attendance record as “Excused” at their discretion.
Rescheduled: A provider or manager has excused an old record and allowed the next attendance date to act as the new date.

IV.F.3.ii Late and Missed Attendance

As seen in FIG. 21, the attendance page 2100 uses these outcomes in a “Last Outcome” column 2128, which shows the outcome from the most recently scheduled collection for each listed patient, and an “Attendance Issues” column 2132, which shows any negative outcomes (e.g., Late, Overdue, and Missed) that have occurred more than a predetermined number of times (e.g., 2 or 3, etc.) over a predetermined time period (e.g., entire patient history, within the last year, within the last 3 months, etc.). The RS&T system is configured to automatically populate the “Attendance Issues” column 2132. In this example, Late and Missed attendance outcomes can be changed to “Excused” or “Rescheduled” by an administrator or authorizer user if desired. To do this, the user can navigate to the attendance history (see, e.g., attendance history 1700 of FIG. 17) using the [Attendance] button on the patient's patient-profile page (see, e.g., [Attendance] button 1612 on the patient-profile page 1600 of FIG. 16), and then clicks on the desired one of the scheduled specimen collections (see, e.g., FIG. 17). This will cause the RS&T system to display an administrator's patient-attendance card for that scheduled specimen collection, such as the patient-attendance card 2200 of FIG. 22. Being the administrators (or authorizer's) version of the patient-attendance card 2200, it includes a “Change Outcome” region 2204 that includes a number of selectable buttons 2204(1) to 2204(5) that the user can select to change the outcome of that particular scheduled specimen collection. As can be seen in FIG. 22, the options for changing the outcome are “Rescheduled” 2204(1), “Excused” 2204(2), “Overdue” 2204(3), “Late” 2204(4), and “Missed” 2204(5).

IV.F.3.iii Reviewing a Patient-Information Card

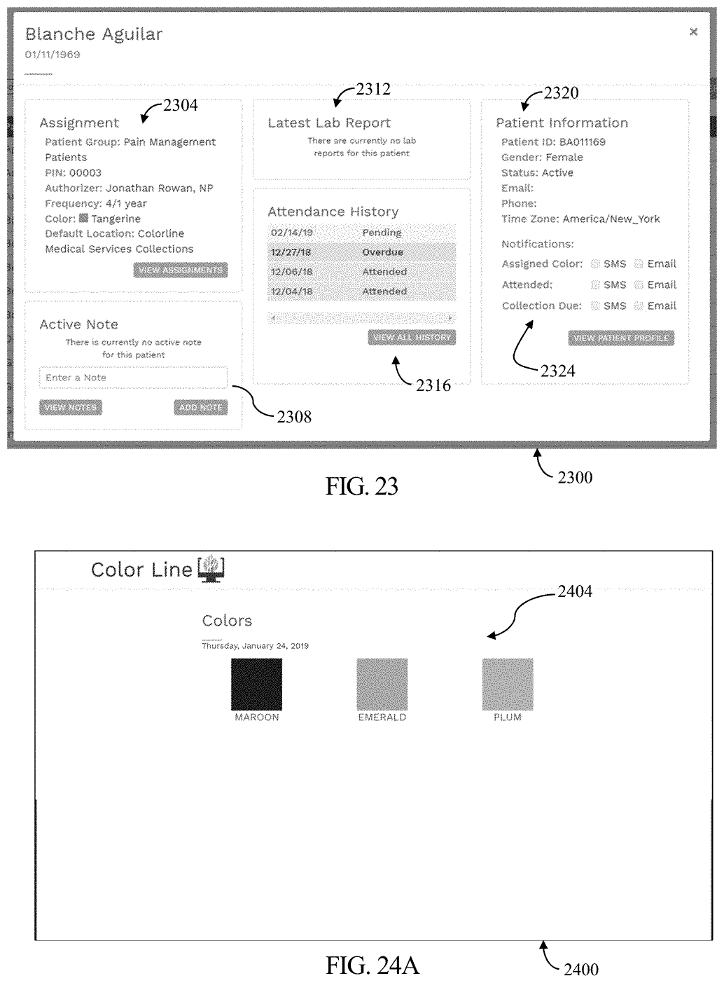

The attendance page 2100 (FIG. 21) provides a user with access to an all-inclusive modal patient-information card, such as patient-information card 2300 of FIG. 23, to allow the user to review patient information all in one place. In this embodiment, the user can access this patient-information card 2300 by clicking on the hyperlinked name 2136 (only one labeled for convenience) of desired patient on the Attendance page 2100 (FIG. 21). This is helpful in viewing all associated factors with a patient's treatment. As seen in FIG. 23, the patient-information card 2300 displays assignment information (region 2304), patient notes (region 2308), laboratory reports (region 2312), attendance history (region 2316), patient demographic information (region 2320), and notification preferences (region 2324). The patient-information card 2300 also includes links to the assignment and attendance history, as well as the patient note archive.

IV.G Accessing Daily Colors

Patients on the example RS&T system have a variety of ways to find out whether or not the RS&T system has “called” their color such that they need to visit a collection location.

IV.G.1 Colors Page

One reliable way to get daily colors is to use the RS&T system's Colors page, such as the example Colors page 2400 of FIG. 24A. A patient can access the Colors page 2400 at any time to see if the RS&T system has populated its current-day color palate 2404 with the patient's color. In this example, a user uses a previously provided URL or corresponding link to access the Colors page 2400 via a suitable UI, such as a web browser (not shown). Before the RS&T system serves the Colors page 2400 to the user, it first displays PIN-entry window, such as PIN-entry window 2408 of FIG. 24B, having a PIN field 2412 where the patient enters the PIN assigned to their patient group (see above). The RS&T system then uses that PIN to access the colors of the day for that patient group and the displays the Colors page 2400 with those colors of the day displayed in the current-day color palate 2404.

IV.G.2 Colors by Phone

Patients can also call using a telephone to get daily colors. In order to access the current-day colors by phone, the patient will call a phone number previously provided to them, enter their PIN when prompted by an automated message system, and, in response thereto, receive a voice list of the daily color palette.

IV.G.3 Colors by Notifications

Patients can be notified that their color has been called by SMS text message or email if this has been set up on their patient profile, such as via the “Notification Preferences” region 1620 of the patient-profile page 1600 of FIG. 16. While these notifications will still be random because of the TFT flag schedule randomization algorithm that the RS&T system uses to generate the colors of the day, for some patients this method of notification is less desirable than both of the Colors page and colors by phone notifications of sections IV.G.1 and IV.G.2, above, that require active involvement by the patient. With the Colors page and the colors by phone methods, the patient is required to either access the Colors page or make a phone call every day that their collection location is open. This additional patient involvement can be part of the patient's recovery plan and experience.

V. Example Computing System

The software for any one or more of the aspects of an RS&T system of the present disclosure, such as RS&T system 100 of FIGS. 1A and 1B, may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

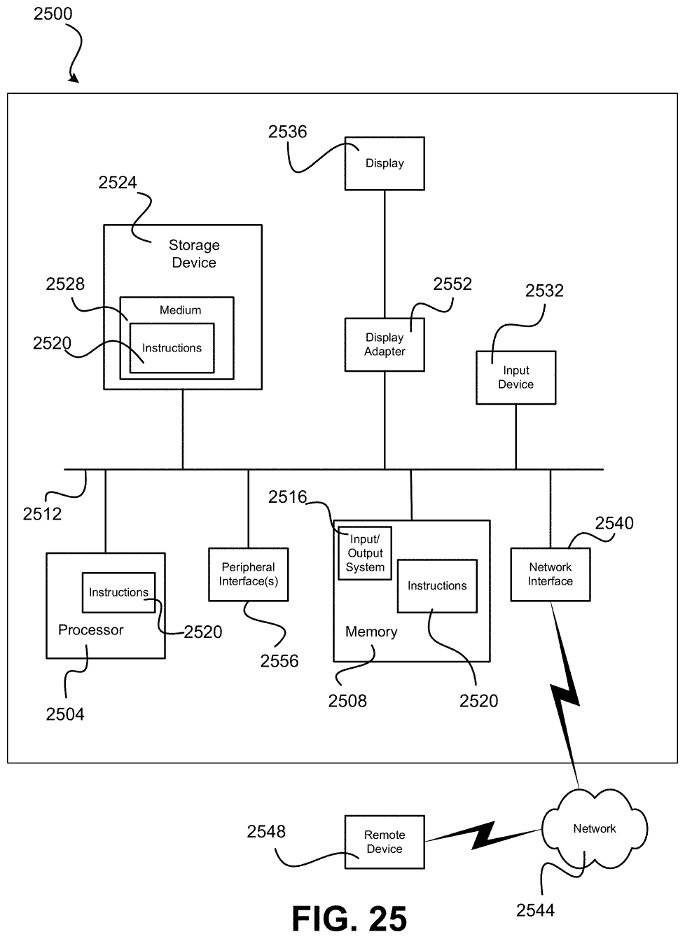

FIG. 25 shows a diagrammatic representation of one embodiment of a computing device in the exemplary form of a computer system 2500 within which a set of instructions for causing a control system, such as the X system of FIG. Y, to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 2500 includes a processor 2504 and a memory 2508 that communicate with each other, and with other components, via a bus 2512. Bus 2512 may include any of several types of bus structures including, but not limited to, a memory bus, a memory controller, a peripheral bus, a local bus, and any combinations thereof, using any of a variety of bus architectures.

Memory 2508 may include various components (e.g., machine-readable media) including, but not limited to, a random access memory component, a read only component, and any combinations thereof. In one example, a basic input/output system 2516 (BIOS), including basic routines that help to transfer information between elements within computer system 2500, such as during start-up, may be stored in memory 2508. Memory 2508 may also include (e.g., stored on one or more machine-readable media) instructions (e.g., software) 2520 embodying any one or more of the aspects and/or methodologies of the present disclosure. In another example, memory 2508 may further include any number of program modules including, but not limited to, an operating system, one or more application programs, other program modules, program data, and any combinations thereof.

Computer system 2500 may also include a storage device 2524. Examples of a storage device (e.g., storage device 2524) include, but are not limited to, a hard disk drive, a magnetic disk drive, an optical disc drive in combination with an optical medium, a solid-state memory device, and any combinations thereof. Storage device 2524 may be connected to bus 2512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 2524 (or one or more components thereof) may be removably interfaced with computer system 2500 (e.g., via an external port connector (not shown)). Particularly, storage device 2524 and an associated machine-readable medium 2528 may provide nonvolatile and/or volatile storage of machine-readable instructions, data structures, program modules, and/or other data for computer system 2500. In one example, software 2520 may reside, completely or partially, within machine-readable medium 2528. In another example, software 2520 may reside, completely or partially, within processor 2504.

Computer system 2500 may also include an input device 2532. In one example, a user of computer system 2500 may enter commands and/or other information into computer system 2500 via input device 2532. Examples of an input device 2532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 2532 may be interfaced to bus 2512 via any of a variety of interfaces (not shown) including, but not limited to, a serial interface, a parallel interface, a game port, a USB interface, a FIREWIRE interface, a direct interface to bus 2512, and any combinations thereof. Input device 2532 may include a touch screen interface that may be a part of or separate from display 2536, discussed further below. Input device 2532 may be utilized as a user selection device for selecting one or more graphical representations in a graphical interface as described above.

A user may also input commands and/or other information to computer system 2500 via storage device 2524 (e.g., a removable disk drive, a flash drive, etc.) and/or network interface device 2540. A network interface device, such as network interface device 2540, may be utilized for connecting computer system 2500 to one or more of a variety of networks, such as network 2544, and one or more remote devices 2548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 2544, may employ a wired and/or a wireless mode of communication. In general, any network topology may be used. Information (e.g., data, software 2520, etc.) may be communicated to and/or from computer system 2500 via network interface device 2540.

Computer system 2500 may further include a video display adapter 2552 for communicating a displayable image to a display device, such as display device 2536. Examples of a display device include, but are not limited to, a liquid crystal display (LCD), a plasma display, a light emitting diode (LED) display, and any combinations thereof. Display adapter 2552 and display device 2536 may be utilized in combination with processor 2504 to provide graphical representations of aspects of the present disclosure. In addition to a display device, computer system 2500 may include one or more other peripheral output devices including, but not limited to, an audio speaker, a printer, and any combinations thereof. Such peripheral output devices may be connected to bus 2512 via a peripheral interface 2556. Examples of a peripheral interface include, but are not limited to, a serial port, a USB connection, a FIREWIRE connection, a parallel connection, and any combinations thereof.

The foregoing has been a detailed description of illustrative embodiments of the invention. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of implementing a randomized testing scheme for notifying a monitored individual of whether or not the monitored individual is due for a monitoring test, wherein the monitored individual has an assigned time-for-testing (TFT) flag selected from a plurality of differing TFT flags and is associated with a testing frequency of a number N times per a time period, the method being executed by a computing system and comprising:
determining a set of testing-available dates corresponding to the time period;
executing a TFT flag schedule randomization algorithm that uses the testing frequency and the set of the testing-available dates to create a randomized TFT flag schedule having one or more instances of a TFT flag indicator corresponding to the assigned TFT flag, wherein the one or more instances are associated with one or more corresponding randomly selected ones of the testing-available dates; and
notifying the monitored individual that the assigned TFT flag has been called.

2. The method of claim 1, wherein notifying the monitored individual includes direct-messaging the monitored individual.

3. The method of claim 2, wherein direct messaging the monitored individual include sending a notification via a network-based messaging service.

4. The method of claim 1, wherein notifying the monitored individual includes populating an anonymized random-testing notification user interface with the assigned TFT flag.

5. The method of claim 4, wherein the anonymized random-testing notification UI comprise a webpage, and the identical access information includes a uniform resource locator.

6. The method of claim 4, wherein the anonymized random-testing notification UI comprises a telephonic message, and the identical access information includes a telephone number.

7. The method of claim 1, wherein the plurality of differing TFT flags comprise a plurality of differing colors.

8. The method of claim 1, further comprising storing the randomized TFT flag schedule in a randomized testing-calendar datastore, the randomized TFT flag schedule containing the one or more instances of the TFT flag indicator.

9. The method of claim 8, further comprising, at a predetermined time on each current day of a plurality of successive testing-available dates:
accessing the randomized testing-calendar datastore to determine if the randomized TFT flag schedule has an instance of the TFT flag indicator assigned to the current day; and
when the randomized TFT flag schedule has an instance of the TFT flag indicator assigned to the current day, populating a anonymized random-testing notification UI with a the TFT flag.

10. The method of claim 9, further comprising, prior to the predetermined time on each current day, automatically executing the TFT flag schedule randomization algorithm to makes any needed updates to the randomized TFT flag schedule.

11. A machine-readable storage medium containing machine executable instructions for performing a method of implementing a randomized testing scheme for notifying a monitored individual of whether or not the monitored individual is due for a monitoring test, wherein the monitored individual has an assigned time-for-testing (TFT) flag selected from a plurality of differing TFT flags and is associated with a testing frequency of a number N times per a time period, the method comprising:
determining a set of testing-available dates corresponding to the time period;
executing a TFT flag schedule randomization algorithm that uses the testing frequency and the set of the testing-available dates to create a randomized TFT flag schedule having one or more instances of a TFT flag indicator corresponding to the assigned TFT flag, wherein the one or more instances are associated with one or more corresponding randomly selected ones of the testing-available dates; and
notifying the monitored individual that the assigned TFT flag has been called.

12. The machine-readable storage medium of claim 11, wherein notifying the monitored individual includes direct-messaging the monitored individual.

13. The machine-readable storage medium of claim 12, wherein direct messaging the monitored individual include sending a notification via a network-based messaging service.

14. The machine-readable storage medium of claim 11, wherein notifying the monitored individual includes populating an anonymized random-testing notification user interface with the assigned TFT flag.

15. The machine-readable storage medium of claim 14, wherein the anonymized random-testing notification UI comprise a webpage, and the identical access information includes a uniform resource locator.

16. The machine-readable storage medium of claim 14, wherein the anonymized random-testing notification UI comprises a telephonic message, and the identical access information includes a telephone number.

17. The machine-readable storage medium of claim 11, wherein the plurality of differing TFT flags comprise a plurality of differing colors.

18. The machine-readable storage medium of claim 11, further comprising storing the randomized TFT flag schedule in a randomized testing-calendar datastore, the randomized TFT flag schedule containing the one or more instances of the TFT flag indicator.

19. The machine-readable storage medium of claim 18, further comprising, at a predetermined time on each current day of a plurality of successive testing-available dates:
accessing the randomized testing-calendar datastore to determine if the randomized TFT flag schedule has an instance of the TFT flag indicator assigned to the current day; and
when the randomized TFT flag schedule has an instance of the TFT flag indicator assigned to the current day, populating a anonymized random-testing notification UI with a the TFT flag.

20. The machine-readable storage medium of claim 19, further comprising, prior to the predetermined time on each current day, automatically executing the TFT flag schedule randomization algorithm to makes any needed updates to the randomized TFT flag schedule.
